(19) United States
(12) Patent Application Publication
(10) Pub. No.: US 2002/0127652 A1
(43) Pub. Date: Sep. 12, 2002

(54) Follicle stimulating hormones

(76) Inventors: Hans Thalsgard Schambye, Frederiksberg (DK); Kim Vilbour Andersen, Copenhagen (DK); Bart van den Hazel, Copenhagen (DK); Jesper Christiansen, Lyngby (DK); Claus Bekker Jeppesen, Nivaa (DK)

Correspondence Address: LAW OFFICES OF JONATHAN ALAN QUINE, P O BOX 458, ALAMEDA, CA, 94501

(21) Appl. No.: 09/780,933

(22) Filed: Feb. 9, 2001

Related U.S. Application Data
(60) Provisional application No. 60/184,035, filed on Feb. 22, 2000.
(60) Provisional application No. 60/225,558, filed on Aug. 16, 2000.

(30) Foreign Application Priority Data
Feb. 11, 2000 (DK) PA2000 00220
Jul. 14, 2000 (DK) PA2000 01092

(51) Int. Cl. C07H 21/04; A61K 38/24; C07K 14/59

(52) U.S. Cl. 435/69.4; 514/12; 530/397; 536/23.5

(57) ABSTRACT

Heterodimeric polypeptide conjugates exhibiting FSH activity, comprising a dimeric polypeptide comprising an FSH-α subunit and an FSH-β subunit, wherein at least one of the FSH-α and FSH-β subunits differs from the corresponding wildtype subunit in that at least one amino acid residue acid residue comprising an attachment group for a non-polypeptide moiety has been introduced or removed, and having at least one non-polypeptide moiety bound to an attachment group of at least one of said subunits are provided. Preferably, at least one attachment group, e.g., an N- or O-glycosylation site or an attachment site for a polymer molecule such as polyethylene glycol, has been introduced, e.g., at an N-terminal. The polypeptide conjugates exhibit improved properties, in particular an increased half-life, compared to human FSH.

### CROSS-REFERENCES TO RELATED APPLICATIONS

This application claims priority to and benefit of the following international and United States Patent Applications: Danish Patent Application PA 2000 00220, filed Feb. 11, 2000; U.S. Patent Provisional Application No. 60/184,035, filed Feb. 22, 2000; Danish Patent Application PA 2000 01092, filed Jul. 14, 2000; and U.S. Provisional Application No. 60/225,558, filed Aug. 16, 2000, the specifications of which are incorporated herein in their entirety for all purposes.

### COPYRIGHT NOTICE

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to new polypeptides and polypeptide conjugates exhibiting follicle stimulating hormone (FSH) activity, to methods for preparing such polypeptides and conjugates, and to the use of such polypeptides and conjugates in therapy, in particular in the treatment of infertility.

### BACKGROUND OF THE INVENTION

Follicle Stimulating Hormone (FSH) is a dimeric hormone consisting of an α subunit and a β subunit. The α subunit is common to the glycoprotein hormone family, which in addition to FSH includes chorionic gonadotropin (CG), thyroid stimulating hormone (TSH), and luteinizing hormone (LH). The β subunit is specific to FSH. The human wildtype α subunit is a 92 amino acid glycoprotein, the amino acid sequence of which is shown in SEQ ID NO:2. Said subunit is referred to herein as hFSH-α. The human wildtype β subunit is a 111 amino acid glycoprotein that has the amino acid shown in SEQ ID NO:4. This subunit is referred to herein as hFSH-β.

Human FSH (hFSH) has been isolated from pituitary glands and from post-menopausal urine (EP 322 438) and has been produced recombinantly in mammalian cells (U.S. Pat Nos. 5,639,640, 5,156,957, 4,923,805, 4,840,896, 5,767,251, EP 211,894 and EP 521,586). The latter references also disclose the hFSH-β gene. U.S. Pat. No. 5,405,945 discloses a modified human α subunit gene comprising only one intron.

U.S. Pat. Nos. 4,589,402 and 4,845,077 disclose purified hFSH which is free of LH and the use thereof for in vitro fertilization. EP 322 438 discloses a protein with at least 6200 U/mg FSH activity which is substantially free of LH activity, and wherein the FSH α subunit and β subunit, respectively, can be wildtype or specified truncated forms thereof.

Liu et al., J Biol Chem 1993, 15;268(2):21613-7, Grossmann et al., Mol Endocrinol 1996 10(6): 769-79, Roth and Dias (Mol Cell Endocribol 1995 1; 109(2): 143-9, Valove et al., Endocrinology 1994; 135(6):2657-61 ,Yoo et al., J Biol Chem 1993 25; 268(18): 13034-42), U.S. Pat. No. 5,508,261 and Chappel et al., 1998, Human Reproduction, 13(3): 18-35 disclose various structure-function relationship studies and identify amino acid residues involved in receptor binding and activation and in dimerization of FSH.

It has been found that glycosylation of FSH-α and FSH-β is essential for receptor signal transduction. hFSH-α comprises two N-glycosylation sites at the asparagines located at position 52 and 78, whereas hFSH-β comprises two N-glycosylation sites at the asparagines located at positions 7 and 24. The importance of the various N-glycosylation sites for the binding and signal-transducing activities of FSH are discussed, inter alia, by Valove et al., Endocrinology 1994; 135(6):2657-61 and Flack et al., J Biol Chem 1994 13;269(19):14015-20.

Galway et al., Endocrinology 1990; 127(1):93-100 demonstrate that FSH variants produced in a N-acetylglucosamine transferase-I CHO cell line or a CHO cell line defective in sialic acid transport are as active as FSH secreted by wildtype cells or purified pituitary FSH in vitro, but lacked in vivo activity, presumably due to rapid clearance of the inadequately glycosylated variants in serum. D'Antonio et al., Human Reprod 1999; 14(5):1160-7 describe various FSH isoforms circulating in the blood stream. The isoforms have identical amino acid sequences, but differ in their extent of post-translational modification. It was found that the less acidic isoform group had a faster in vivo clearance as compared with the acidic isoform group, possibly due to differences in the sialic acid content between the isoforms.

U.S. Pat. No. 5,087,615 discloses a method for stimulating follicle development and ovulation in a female patient by administering FSH to said patient during the follicular phase of the ovulatory cycle, the improvement comprising initially adminstering a first FSH isoform having a relatively long plasma half-life and subsequently administering a second FSH isoform having a shorter plasma half-life.

Bishop et al. Endocrinology 1995; 136(6):2635-40 conclude that circulatory half-life appears to be the primary determinant of in vivo activity.

Attempts have been made to prolong the serum half-life of FSH. U.S. Pat. Nos. 5,338,835 and 5,585,345 disclose a modified FSH-β subunit extended at the C-terminal Glu with the carboxy terminal portion (CTP) region of hCG (the region consisting of the amino acid sequence which occurs from positions 112-118 to 145, and comprising four 0-linked glycosylation sites located at positions 121, 127, 132 and 138). The resulting modified subunit is stated to have the biological activity of native FSH, but a prolonged circulating half-life. U.S. Pat. No. 5,405,945 discloses that the carboxy terminal portion of the CG β subunit or a variant thereof has significant effects on the clearance of CG, FSH, and LH.

U.S. Pat. No. 5,883,073 discloses single-chain proteins comprised of two α-subunits with agonist or antagonist activity for CG, TSH, LH and FSH.

U.S. Pat. No. 5,508,261 discloses heterodimeric polypeptides having binding affinity to LH and FSH receptors comprising a glycoprotein hormone α subunit and a non-naturally occurring β subunit polypeptide, wherein the β subunit polypeptide is a chain of amino acids comprising four joined subsequences, each of which is selected from a list of specific sequences.

U.S. Pat. No. 5,567,422 and WO 98/32466 mention FSH among a vast number of other therapeutic proteins that can be PEGylated.

Currently, FSH is used therapeutically to stimulate the growth and maturation of ovarian follicles in infertile women. In particular, FSH is used in connection with in vitro fertilization as well as for the treatment of anovulatory women, with anovulatory syndrome or luteal phase deficiency. However, one problem encountered in current FSH treatment is the fairly short in vivo half-life of FSH requiring frequent, usually daily administration of the product. The frequent administration is very inconvenient for the patient and results in high fluctuations of FSH activity in the blood stream, which can cause inadequate maturation of the follicles.

Therefore, a clinical need exists for a product which provides part or all of the therapeutically relevant effects of FSH, and which can be administered at less frequent intervals as compared to currently available FSH product, and which preferably provides a more stable level of circulating FSH activity as compared to that obtainable by current treatment. The present invention provides such products as well as the means of making such products.

### SUMMARY OF THE INVENTION

The present invention relates to polypeptide conjugates exhibiting FSH activity and methods for their preparation and their use in medical treatment.

Accordingly, in a first aspect, the invention relates to a heterodimeric polypeptide conjugate exhibiting FSH activity, comprising i) a dimeric polypeptide comprising an FSH-α subunit and an FSH-β subunit, wherein at least one of said FSH-α and FSH-β subunits differs from the corresponding wildtype subunit in that at least one amino acid residue acid residue comprising an attachment group for a non-polypeptide moiety has been introduced or removed, and ii) at least one non-polypeptide moiety bound to an attachment group of at least one of said subunits.

In another aspect, the invention relates to a heterodimeric polypeptide conjugate exhibiting FSH activity, comprising i) a dimeric polypeptide comprising an FSH-α subunit and an FSH-β subunit, wherein the amino acid sequence of at least one of said FSH-α and FSH-β subunits differs from that of the corresponding wildtype subunit in that at least one N-glycosylation site has been introduced, and ii) at least one oligosaccharide moiety bound to an N-glycosylation site of at least one of said subunits.

In a further aspect, the invention relates to a heterodimeric polypeptide conjugate exhibiting FSH activity, comprising a dimeric polypeptide comprising FSH-α and FSH-β subunits, wherein at least one of said FSH-α and FSH-β subunits comprises, relative to the corresponding wildtype subunit, at least one introduced N- or O-glycosylation site at the N-terminal thereof, said at least one introduced glycosylation site being glycosylated.

In the above aspects, the corresponding wildtype subunits are preferably hFSH-α and hFSH-β, respectively.

Another aspect of the invention relates to a heterodimeric polypeptide conjugate exhibiting FSH activity, comprising a dimeric polypeptide comprising an FSH-α subunit and an FSH-β subunit, wherein at least one of said FSH-α and FSH-β subunits comprises a polymer molecule bound to the N-terminal thereof.

In a further aspect, the invention relates to modified FSH-α and modified FSH-β polypeptides that can be used as intermediate products for the preparation of a conjugate with a polymer molecule.

In still further aspects, the invention relates to methods for preparing a conjugate or a polypeptide of the invention, including nucleotide sequences and expression vectors encoding a polypeptide or a conjugate of the invention.

In yet other aspects, the invention relates to a composition comprising a conjugate or polypeptide of the invention and methods of treating a mammal with such composition. In particular, the polypeptide, conjugate or composition of the invention can be used to treat infertility.

### BRIEF DESCRIPTION OF THE FIGURES

FIG. 1 shows a sequence alignment of human FSH to the structural part of two published structures of human chorionic gonadotropin.

### DETAILED DISCUSSION

Definitions

In the context of the present application and invention the following definitions apply:

The term “conjugate” is intended to indicate a heterogeneous molecule formed by the covalent attachment of one or more polypeptides to one or more non-polypeptide moieties such as polymer molecules, oligosaccharide moieties, lipophilic compounds, carbohydrate moieties or organic derivatizing agents. The term covalent attachment means that the polypeptide and the non-polypeptide moiety are either directly covalently joined to one another, or else are indirectly covalently joined to one another through an intervening moiety or moieties, such as a bridge, spacer, or linkage moiety or moieties. Preferably, the conjugate is soluble at relevant concentrations and conditions, i.e., soluble in physiological fluids such as blood. The term “non-conjugated polypeptide” can be used about the polypeptide part of the conjugate.

The term “polypeptide” can be used interchangeably herein with the term “protein.” Further, the terms “polypeptide” and “protein” are generally used herein for the sake of simplicity to refer to the heterodimeric FSH polypeptides/proteins and conjugates of the invention, even though these proteins strictly speaking comprise a dimer of the α and β polypeptide subunits. The individual subunits are referred to herein as FSH-α and FSH-β, respectively, so that it is clear from the context whether reference is made to the dimeric hormone or to one of the subunits.

The “polymer molecule” is a molecule formed by covalent linkage of two or more monomers, wherein none of the monomers is an amino acid residue, except where the polymer is human albumin or another abundant plasma protein. The term “polymer” can be used interchangeably with the term “polymer molecule.” The term is intended to cover carbohydrate molecules attached by in vitro glycosylation. Carbohydrate molecules attached by in vivo glycolsylation, such as N- or O-glycosylation (as further described below) are referred to herein as “an oligosaccharide moiety.” Except where the number of polymer molecules is expressly indicated, every reference to “a polymer,” “a polymer molecule,” “the polymer” or “the polymer molecule” contained in polypeptide of the invention or otherwise used in the present invention shall be a reference to one or more polymer molecule(s).

The term “attachment group” is intended to indicate an amino acid residue group of the polypeptide capable of coupling to the relevant non-polypeptide moiety. For instance, for polymer conjugation to PEG, a frequently used attachment group is the ε-amino group of lysine or the N-terminal amino group. Other polymer attachment groups include a free carboxylic acid group (e.g., that of the C-terminal amino acid residue or of an aspartic acid or glutamic acid residue), suitably activated carbonyl groups, oxidized carbohydrate moieties and mercapto groups. Useful attachment groups and their matching non-peptide moieties are apparent from the table below.

| | | | Conjugation | |
|---|---|---|---|---|
| Attachment | | Examples of non- | method/- | |
| group | Amino acid | peptide moiety | Activated PEG | Reference |
| —NH₂ | N-terminal, | Polymer, e.g., | mPEG-SPA | Shearwater Inc. |
| | Lys, His, Arg | PEG, with amide | Tresylated | Delgado et al., |
| | | or imine group | mPEG | critical reviews in |
| | | | | Therapeutic Drug |
| | | | | Carrier Systems |
| | | | | 9(3,4):249-304 |
| | | | | (1992) |
| —COOH | C-term, Asp, | Polymer, e.g., | mPEG-Hz | Shearwater Inc. |
| | Glu | PEG, with ester or | | |
| | | amide group | | |
| | | Oligosaccharide | In vitro coupling | |
| | | moiety | | |
| —SH | Cys | Polymer, e.g., | PEG- | Shearwater Inc. |
| | | PEG, with | vinylsulphone | Delgado et al., |
| | | disulfide, | PEG-maleimide | critical reviews in |
| | | maleimide or vinyl | | Therapeutic Drug |
| | | sulfone group | | Carrier Systems |
| | | Oligosaccharide | In vitro coupling | 9(3,4):249-304 |
| | | moiety | | (1992) |
| —OH | Ser, Thr, —OH, | Oligosaccharide | In vivo O-linked | |
| | Lys | moiety | glycosylation | |
| | | PEG with ester, | | |
| | | ether, carbamate, | | |
| | | carbonate | | |
| —CONH₂ | Asn as part of | Oligosaccharide | In vivo N- | |
| | an N-glyco- | moiety | glycosylation | |
| | sylation site | Polymer, e.g., PEG | | |
| Aromatic | Phe, Tyr, Trp | Oligosaccharide | In vitro coupling | |
| residue | | moiety | | |
| —CONH₂ | Gln | Oligosaccharide | In vitro coupling | Yan and Wold, |
| | | moiety | | Biochemistry, 1984, |
| | | | | Jul 31; 23(16): 3759- |
| | | | | 65 |
| Aldehyde | Oxidized | Polymer, e.g., | PEGylation | Andresz et al., 1978, |
| Ketone | oligo- | PEG, | | Makromol. Chem. |
| | saccharide | PEG-hydrazide | | 179:301, WO |
| | | | | 92/16555, WO |
| | | | | 00/23114 |
| Guanidino | Arg | Oligosaccharide | In vitro coupling | Lundblad and |
| | | moiety | | Noyes, Chemical |
| | | | | Reagents for Protein |
| | | | | Modification, CRC |
| | | | | Press Inc., Florida, |
| | | | | USA |
| Imidazole | His | Oligosaccharide | In vitro coupling | As for guanidine |
| ring | | moiety | | |

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue which optionally can be identical to X′ and which preferably is different from proline, N is asparagine, and S/T/C is either serine, threonine or cysteine, preferably serine or threonine, and most preferably threonine). Although the asparagine residue of the N-glycosylation site is where the oligosaccharide moiety is attached during glycosylation, such attachment cannot be achieved unless the other amino acid residues of the N-glycosylation site are present. Accordingly, when the non-peptide moiety is an oligosaccharide moiety and the conjugation is to be achieved by N-glycosylation, the term “amino acid residue comprising an attachment group for the non-peptide moiety” as used in connection with alterations of the amino acid sequence of the polypeptide of interest is to be understood as meaning that one or more amino acid residues constituting an N-glycosylation site are to be altered in such a manner that either a functional N-glycosylation site is introduced into the amino acid sequence or removed from said sequence.

In the present application, amino acid names and atom names (e.g., CA, CB, NZ, N, O, C, etc.) are used as defined by the Protein DataBank (PDB) (www.pdb.org), which is based on the IUPAC nomenclature (IUPAC Nomenclature and Symbolism for Amino Acids and Peptides (residue names, atom names etc.), Eur. J. Biochem., 138, 9-37 (1984) together with their corrections in Eur. J. Biochem., 152, 1 (1985). The term “amino acid residue” is primarily intended to indicate an amino acid residue contained in the group consisting of the 20 naturally occurring amino acids, i.e., alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues.

The terminology used for identifying amino acid positions/substitutions is illustrated as follows: E9(a) indicates position number 9 occupied by a glutamic acid residue in the amino acid sequence shown in SEQ ID NO:2. E9(a)N indicates that said glutamic acid residue has been substituted by an asparagine residue. Unless otherwise indicated, the numbering of amino acid residues made herein is made relative to the amino acid sequence shown in SEQ ID NO:2 (for FSH-α, indicated by “(a)”) or SEQ ID NO:4 (for FSH-β, indicated by “(b)”). Multiple substitutions are indicated with a “+,” e.g., M109(b)N+E111(b)S/T means an amino acid sequence which comprises substitution of the methionine residue in position 109 of FSH-β by an asparagine residue and substitution of the glutamic acid residue in position 111 in FSH-β by a serine or a threonine residue.

The term “nucleotide sequence” is intended to indicate a consecutive stretch of two or more nucleotide molecules. The nucleotide sequence can be of genomic, cDNA, RNA, semisynthetic, synthetic origin, or any combination thereof.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

“Cell,” “host cell,” “cell line” and “cell culture” are used interchangeably herein and all such terms should be understood to include progeny resulting from growth or culturing of a cell. “Transformation” and “transfection” are used interchangeably to refer to the process of introducing DNA into a cell.

“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

The term “introduce” refers to introduction of an amino acid residue comprising an attachment group for a non-polypeptide moiety, either by substitution of an existing amino acid residue or by insertion of an additional amino acid residue. The term “remove” refers to removal of an amino acid residue comprising an attachment group for a non-polypeptide moiety, either by substitution of the amino acid residue to be removed by another amino acid residue or by deletion (without substitution) of the amino acid residue to be removed.

When substitutions are performed in relation to a parent polypeptide, they are preferably “conservative substitutions,” in other words substitutions performed within groups of amino acids with similar characteristics, e.g., small amino acids, acidic amino acids, polar amino acids, basic amino acids, hydrophobic amino acids and aromatic amino acids.

Preferred substitutions in the present invention can in particular be chosen from among the conservative substitution groups listed in the table below.

Conservative Substitution Groups

| | | | | |
|---|---|---|---|---|
| 1 | Alanine (A) | Glycine (G) | Serine (S) | Threonine |
| | | | | (T) |
| 2 | Aspartic acid (D) | Glutamic acid (E) | | |
| 3 | Asparagine (N) | Glutamine (Q) | | |
| 4 | Arginine (R) | Histidine (H) | Lysine (K) | |
| 5 | Isoleucine (I) | Leucine (L) | Methionine (M) | Valine (V) |
| 6 | Phenylalanine (F) | Tyrosine (Y) | Tryptophan (W) | |

The term “immunogenicity” as used in connection with a given substance is intended to indicate the ability of the substance to induce a response from the immune system. The immune response can be a cell or antibody mediated response (see, e.g., Roitt: Essential Immunology (8^{th} Edition, Blackwell) for further definition of immunogenicity). Normally reduced antibody reactivity will be an indication of a reduced immunogenicity. The reduced immunogenicity can be determined by use of any suitable method known in the art, e.g., in vivo or in vitro.

The term “functional in vivo half-life” is used in its normal meaning, i.e., the time at which 50% of the biological activity of the polypeptide or conjugate is still present in the body/target organ, or the time at which the activity of the polypeptide or conjugate is 50% of the initial value. As an alternative to determining functional in vivo half-life, “serum half-life” can be determined, i.e., the time at which 50% of the dispensed polypeptide or conjugate molecules is still present in the circulation/plasma/bloodstream. The magnitude of serum half-life is usually a good indication of the magnitude of functional in vivo half-life. Alternative terms to serum half-life include “plasma half-life,” “circulating half-life,” “serum clearance,” “plasma clearance” and “clearance half-life.” The polypeptide or conjugate is cleared by the action of one or more of the kidney, reticuloendothelial systems (RES), spleen or liver, by FSH-receptor-mediated elimination, or by specific or non-specific proteolysis. Normally, clearance depends on size (relative to the cutoff for glomerular filtration), charge, attached carbohydrate chains, and the presence of cellular receptors for the protein. The functional in vivo half-life and the serum half-life can be determined by any suitable method known in the art as further discussed in the Examples section hereinafter.

The term “increased” as used about the functional in vivo half-life or serum half-life is used to indicate that the relevant half-life of the conjugate or polypeptide is statistically significantly increased relative to that of a reference molecule, such as a non-conjugated rhFSH (recombinant human FSH), e.g., Gonal-F® (available from Serono) or Puregon® (available from Organon), as determined under comparable conditions. For instance, the relevant half-life can be increased by at least about 25%, such as by at least about 50%, e.g., by at least about 100%, 200% or 500%.

The term “renal clearance” is used in its normal meaning to indicate any clearance taking place by the kidneys, e.g., by glomerular filtration, tubular excretion or tubular elimination. Renal clearance depends on physical characteristics of the conjugate, including size (diameter), symmetry, shape/rigidity and charge. Reduced renal clearance can be established by any suitable assay, e.g., an established in vivo assay. Typically, renal clearance is determined by administering a labelled (e.g., radioactive or fluorescent labelled) polypeptide conjugate to a patient and measuring the label activity in urine collected from the patient. Reduced renal clearance is determined relative to a corresponding reference polypeptide, e.g., the corresponding non-conjugated polypeptide, a non-conjugated corresponding wild-type polypeptide or another conjugated polypeptide (such as a conjugated polypeptide not according to the invention), under comparable conditions.

In some cases, it will be preferred to obtain a clearance that is only slightly reduced (i.e., total clearance by renal clearance, receptor-mediated clearance and/or other clearance mechanisms), e.g., to increase the in vivo half-life from about 24 hours to about 3-4 days, while in other cases a longer half-life of e.g., about 6-7 days will be desired. As will be explained in further detail below, the number and size of such polymer molecules can be adapted in order to obtain a desired clearance, as well as other desired properties, suitable for a given clinical indication. Preferably, the conjugate of the invention has a reduced clearance of at least about 50%, such as least about 75% or at least about 90%, as compared to the corresponding non-conjugated polypeptide (such as hFSH or rhFSH) as determined under comparable conditions.

Generally, activation of the receptor is coupled to receptor-mediated clearance (RMC) such that binding of a polypeptide to its receptor without activation does not lead to RMC, while activation of the receptor leads to RMC. The clearance is due to internalisation of the receptor-bound polypeptide with subsequent lysosomal degradation. Reduced RMC can therefore be achieved by designing the conjugate so as to be able to bind and activate a sufficient number of receptors to obtain optimal in vivo biological response and avoid activation of more receptors than required for obtaining such response, e.g., by substitution, polymer conjugation or other modification of one or more amino acid residues located at or near a receptor-binding site. This can be reflected in reduced in vitro bioactivity and/or increased off-rate.

The term “FSH-α” is intended to indicate a polypeptide having qualitatively similar functions or activities as the corresponding wildtype FSH α subunit, including the capability of forming a dimeric polypeptide with an FSH-β subunit (FSH-β), which dimeric polypeptide exhibits FSH activity. Alternatively used terms include “FSH-α polypeptide,” “FSH-α subunit,” and “modified FSH-α.” Analogously, the term “FSH-β” is intended to indicate a polypeptide having qualitatively similar functions or activities as the corresponding wildtype FSH β subunit, including the capability of dimerizing with FSH-α and thereby forming a dimeric polypeptide exhibiting FSH activity. Alternatively used terms include “FSH-β polypeptide,” “FSH-β subunit,” and “modified FSH-β.”

The term “exhibiting FSH activity” is intended to indicate that the conjugate or polypeptide has one or more of the functions of wildtype FSH, in particular hFSH, including the capability of binding to and activating an FSH receptor. The FSH activity is conveniently assayed using the in vitro activity assay described in the Examples section below. The conjugate or polypeptide “exhibiting” FSH activity is considered to have such activity when it displays a measurable function, e.g., a measurable activity. The dimeric polypeptide exhibiting FSH activity can also be termed “FSH molecule” herein.

Conjugate of the Invention

As stated above, in a first aspect, the invention relates to a polypeptide conjugate exhibiting FSH activity, comprising i) a polypeptide comprising FSH-α and FSH-β subunits, wherein at least one of the FSH-α and FSH-β subunits differs from the corresponding wildtype subunit in at least one introduced or removed amino acid residue comprising an attachment group for non-polypeptide moiety, and ii) a non-polypeptide moiety bound to an attachment group of the polypeptide. Examples of amino acid residues that can be introduced and/or removed are described in further detail in the following sections.

By removing and/or introducing an amino acid residue comprising an attachment group for the non-polypeptide moiety, it is possible to specifically adapt the polypeptide so as to make the molecule more susceptible to conjugation to the non-polypeptide moiety of choice, to optimize the conjugation pattern (e.g., to ensure an optimal distribution of non-polypeptide moieties on the surface of the FSH molecule and to ensure that only the attachment groups intended to be conjugated are present in the molecule) and thereby obtain a new conjugate molecule which has FSH activity and in addition one or more improved properties as compared to FSH molecules available today, in particular increased functional in vivo half-life and/or reduced clearance.

In the conjugate of the invention, one or both of the FSH subunits can be modified according to the invention. For instance, the amino acid sequence of FSH-α can be modified as described herein, whereas FSH-β is unmodified, and vice versa. Alternatively, both of FSH-α and FSH-β can be modified according to the invention.

While the FSH-α and/or FSH-β can be of any origin, it is in particular of mammalian origin, and preferably of human origin. Accordingly, the corresponding wildtype subunits referred to above are preferably hFSH-α and hFSH-β, respectively, with the amino acid sequences shown in SEQ ID NO:2 and 4.

In a preferred embodiment, one difference between the amino acid sequence of FSH-α and/or FSH-β and the corresponding wildtype sequence is that at least one and preferably more, e.g., 1-20, amino acid residues comprising an attachment group for the non-polypeptide moiety have been introduced, by insertion or substitution, into the amino acid sequence. Thereby, properties such as the molecular weight, shape, size and/or charge of the conjugate can be optimised. Preferably, such amino acid residues are introduced in positions occupied by an amino acid residue having more than 25%, more preferably more than 50%, such as more than 75% of its side chain exposed at the surface of the molecule.

The term “one difference” as used in the present application is intended to allow for additional differences being present. Accordingly, in addition to the specified amino acid difference, other amino acid residues than those specified can be mutated.

In one embodiment, one difference between the amino acid sequence of FSH-α and/or FSH-β and that of the corresponding wildtype polypeptide is that at least one and possible more, e.g., 1-15, amino acid residues comprising an attachment group for the non-polypeptide moiety have been removed, by substitution or deletion, from the amino acid sequence. The amino acid residue to be removed is preferably one to which conjugation is disadvantageous, e.g., an amino acid residue located at or near a functional site of the polypeptide (since conjugation at such a site can result in inactivation or reduced FSH activity of the resulting conjugate due to impaired receptor recognition). In the present context the term “functional site” is intended to indicate one or more amino acid residues which are essential for or otherwise involved in the function or performance of hFSH, in particular dimerization and/or receptor binding and activation. Such amino acid residues are a part of a functional site. The functional site can be determined by methods known in the art and is preferably identified by analysis of a structure of the polypeptide complexed to a relevant receptor, such as the hFSH receptor.

In another embodiment, the alteration of FSH-α and/or FSH-β embraces removal as well as introduction of amino acid residues comprising an attachment group for the non-polypeptide moiety of choice.

In order to avoid too much disruption of the structure and function of the FSH molecule, the total number of amino acid residues to be altered in accordance with the present invention will typically not exceed 20 for each individual subunit. Preferably, the polypeptide part of the conjugate of the invention or the dimeric polypeptide of the invention comprises an amino acid sequence which differs in a total of 1-20 amino acid residues from the amino acid sequences shown in SEQ ID NO:2 and/or SEQ ID NO:4, such as in 1-15 or 2-12 amino acid residues, e.g., in 3-10 amino acid residues. Thus, normally the polypeptide part of the conjugate or the dimeric polypeptide of the invention comprises an amino acid sequence which in total differs from the amino acid sequences shown in SEQ ID NO:2 and/or SEQ ID NO:4 in 1, 2, 3, 4, 5, 6, 7, 8, 9, 10, 11, 12, 13, 14, 15, 16, 17, 18, 19 or 20 amino acid residues.

The FSH-α and/or FSH-β subunits of the dimeric polypeptide are preferably any of the specific modified FSH-α and/or FSH-β polypeptides disclosed in the subsequent sections having introduced and/or removed amino acid residues comprising an attachment group for the relevant non-polypeptide moiety.

The amino acid residue comprising an attachment group for a non-polypeptide moiety, whether it is removed or introduced, is selected on the basis of the nature of the non-polypeptide moiety of choice and, in most instances, on the basis of the method in which conjugation between the polypeptide and the non-polypeptide moiety is to be achieved. It will be understood that in order to preserve a measurable function of the modified FSH-α and/or FSH-β, amino acid residues to be modified (by deletion or substitution) are selected from those amino acid residues which are not essential for providing a measurable activity. Accordingly, amino acid residues to be modified are different from those required for subunit dimerization and/or receptor binding or activation. The identity of such amino acid residues is described in the art (e.g., references identified in the Background section above) or can be determined by a person skilled in the art using methods known in the art.

In addition to the amino acid alterations disclosed herein aimed at introducing and/or removing attachment sites for the non-polypeptide moiety, the FSH-α and/or FSH-β subunits can comprise other amino acid alterations that need not be related to introduction or removal of attachment sites, i.e., other substitutions, insertions or deletions. These may, for example, include truncation of the N- and/or C-terminus by one or more amino acid residues, or addition of one or more extra residues at the N- and/or C-terminus. Examples of such additional amino acid changes include adding part of or the entire CTP region of hCG to the C-terminus of FSH-α or introducing any other mutation (in particular selected among those reported to enhance FSH activity and/or increase the functional in vivo half-life, cf. the Background of the Invention section herein). In such cases, the amino acid sequence of the basic polypeptide subunits, i.e., the sequence of the subunits excluding any introduced or removed attachment sites, will typically have a degree of homology, compared to the relevant wildtype sequence (normally hFSH-α or hFSH-β), of at least about 80%, more typically at least about 90%, such as at least about 95%. Amino acid sequence homology/identity is conveniently determined from aligned sequences, using e.g., the ClustalW program or from the PFAM families database version 4.0 (http://pfam.wustl.edu/) (Nucleic Acids Res. Jan. 1, 1999; 27(1):260-2) by use of GENEDOC version 2.5 (Nicholas, K. B., Nicholas H. B. Jr., and Deerfield, D. W. II. 1997 GeneDoc: Analysis and Visualization of Genetic Variation, EMBNEW.NEWS 4:14; Nicholas, K. B. and Nicholas H. B. Jr. 1997 GeneDoc: Analysis and Visualization of Genetic Variation).

Preferably, the conjugate of the present invention has one or more improved properties as compared to hFSH, including increased functional in vivo half-life, increased serum half-life, reduced renal clearance, reduced receptor-mediated clearance, reduced immunogenicity and/or an increased bioavailability as compared to rhFSH (e.g., Gonal-F® or Puregon®). Consequently, medical treatment with a conjugate of the invention offers advantages over the currently available FSH compounds, in particular longer duration between injections.

Conjugate of the Invention wherein the Non-polypeptide Moiety is an Oligosaccharide Moiety

It has been found that N-glycosylation is important for FSH activity and also that the extent and type of oligosaccharide moiety attached by in vivo glycosylation is important for functional in vivo half-life of the glycosylated FSH. In order to obtain a different, increased glycosylation it is desirable to introduce at least one glycosylation site. Accordingly, in a preferred aspect, the invention relates to a heterodimeric polypeptide conjugate exhibiting FSH activity comprising a dimeric polypeptide comprising an FSH-α subunit and an FSH-β subunit, wherein the amino acid sequence of at least one of the FSH-α and FSH-β subunits differs from that of the corresponding wildtype subunit in that at least one N-glycosylation site has been introduced, and having at least one oligosaccharide moiety bound to an N-glycosylation site of at least one of the subunits.

A suitable N-glycosylation site can be introduced by introducing, by substitution or insertion, an asparagine residue in a position occupied by an amino acid residue having more than 25% of its side chain exposed at the surface of the polypeptide, which position does not have a proline residue located in position +1 or +3 therefrom. If the amino acid residue located in position +2 is a serine or threonine, no further amino acid substitution is required. However, if this position is occupied by a different amino acid residue, a serine or threonine residue needs to be introduced.

A preferred conjugate, according to this embodiment, is one which comprises a modified FSH-α subunit having an amino acid residue which differs from that of hFSH-α in the introduction of at least one N-glycosylation site by means of a mutation selected from the group consisting of P2(a)N+V4(a)S, P2(a)N+V4(a)T, D3(a)N+Q5(a)S, D3(a)N+Q5(a)T, V4(a)N+D6(a)S, V4(a)N+D6(a)S, D6(a)N+P8(a)S, D6(a)N+P8(a)T, E9(a)N+T11(a)S, E9(a)N, T11(a)N+Q13(a)S, T11(a)N+Q13(a)T, L12(a)N+E14(a)S, L12(a)N+E14(a)T, E14(a)N+P16(a)S, E14(a)N+P16(a)T, P16(a)N+F18(a)S, P16(a)N+F18(a)T, F17(a)N, F17(a)N+S19(a)T, G22(a)N+P24(a)S, G22(a)N+P24(a)T, P24(a)N+L26(a)S, P24(a)N+L26(a)T, F33(a)N+R35(a)S, F33(a)N+R35(a)T, R42(a)N+K44(a)S, R42(a)N+K44(a)T, S43(a)N+K45(a)S, S43(a)N+K45(a)T, K44(a)N+T46(a)S, K44(a)N, K45(a)N+M47(a)S, K45(a)N+M47(a)T, T46(a)N+L48(a)S, T46(a)N+L48(a)T, L48(a)N+Q50(a)S, 148(a)N+Q50(a)T, V49(a)N+K51(a)S, V49(a)N+K51(a)T, Q50(a)N+N52(a)S, Q50(a)N+N52(a)T, V61(a)N+K63(a)S, V61(a)N+K63(a)T, K63(a)N+Y65(a)S, K63(a)N+Y65(a)T, S64(a)N+N66(a)S, S64(a)N+N66(a)T, Y65(a)N+R67(a)S, Y65(a)N+R67(a)T, V68(a)S, V68(a)T, R67(a)N+T69(a)S, R67(a)N, T69(a)N+M71(a)S, T69(a)N+M71(a)T, M71(a)N+G73(a)S, M71(a)N+G73(a)T, G72(a)N+F74(a)S, G72(a)N+F74(a)T, G73(a)N+K75(a)S, G73(a)N+K75(a)T, F74(a)N+V76(a)S, F74(a)N+V76(a)T, K75(a)N+E77(a)S, K75(a)N+E77(a)T, A81(a)N+H83(a)S, A81(a)N+H83(a)T, H83(a)N, T86(a)N+Y88(a)S, T86(a)N+Y88(a)T, Y88(a)N+H90(a)S, Y88(a)N+H90(a)T, Y89(a)N+K91(a)S, Y89(a)N+K91(a)T, H90(a)N and H90(a)N+S92(a)T (positions with more than 25% side chain exposure). Among these possible positions for mutation, more preferred mutations are those where a glycosylation site can be introduced by mutation of a single amino acid residue, i.e., selected from the group consisting of V68(a)S, V68(a)T, E9(a)N, F17(a)N, K44(a)N, R67(a)N, H83(a)N and H90(a)N.

More preferably, a glycosylation site is introduced at a position having more than 50% side chain exposure, i.e., by means of a mutation selected from the group consisting of P2(a)N+V4(a)S, P2(a)N+V4(a)T, D3(a)N+Q5(a)S, D3(a)N+Q5(a)T, V4(a)N+D6(a)S, V4(a)N+D6(a)S, D6(a)N+P8(a)S, D6(a)N+P8(a)T, E9(a)N+T11(a)S, E9(a)N, T11(a)N+Q13(a)S, T11(a)N+Q13(a)T, E14(a)N+P16(a)S, E14(a)N+P16(a)T, P16(a)N+F18(a)S, P16(a)N+F18(a)T, F17(a)N, F17(a)N+S19(a)T, G22(a)N+P24(a)S, G22(a)N+P24(a)T, K45(a)N+M47(a)S, K45(a)N+M47(a)T, T46(a)N+L48(a)S, T46(a)N+L48(a)T, L48(a)N+Q50(a)S, 148(a)N+Q50(a)T, V49(a)N+K51(a)S, V49(a)N+K51(a)T, Q50(a)N+N52(a)S, Q50(a)N+N52(a)T, K63(a)N+Y65(a)S, K63(a)N+Y65(a)T, S64(a)N+N66(a)S, S64(a)N+N66(a)T, V68(a)S, V68(a)T, R67(a)N+T69(a)S, R67(a)N, T69(a)N+M71(a)S, T69(a)N+M71(a)T, G72(a)N+F74(a)S, G72(a)N+F74(a)T, G73(a)N+K75(a)S, G73(a)N+K75(a)T, K75(a)N+E77(a)S, K75(a)N+E77(a)T, T86(a)N+Y88(a)S, T86(a)N+Y88(a)T, Y89(a)N+K91(a)S, Y89(a)N+K91(a)T, H90(a)N, and H90(a)N+S92(a)T. Still more preferably, glycosylation sites are introduced via mutation of a single amino acid residue selected from the group consisting of E9(a)N, F17(a)N, R67(a)N, and H90(a)N.

The FSH-β part of such conjugates with an altered FSH-α subunit can be hFSH-β or any of the modified FSH-β polypeptides described herein.

Alternatively or additionally, the conjugate according to this embodiment comprises a modified FSH-β having an amino acid residue which differs from that of hFSH-β in the introduction of at least one N-glycosylation site by a mutation selected from the group consisting of S2(b)N+E4(b)S, S2(b)N+E4(b)T, E4(b)N+T6(b)S, E4(b)N, L5(b)N+N7(b)S, L5(b)N+L7(b)T, T6(b)N+I8(b)S, T6(b)N+I8(b)T, I8(b)N+I10(b)S, I8(b)S, I8(b)N+I10(b)T, T9(b)N+A11(b)S, T9(b)N+A11(b)T, K14(b)N+E16(b)S, K14(b)N+E16(b)T, F19(b)N+I21(b)S, F19(b)N+I21(b)T, I21(b)N+I23(b)S, I21(b)N+I23(b)T, S22(b)N+N24(b)S, S22(b)N+N24(b)T, Y31(b)N+Y33(b)S, Y31(b)N+Y33(b)T, Y33(b)N+R35(b)S, Y33(b)N+R35(b)T, R35(b)N+L37(b)S, R35(b)N+L37(b)T, D36(b)N+V38(b)S, D36(b)N+V38(b)T, L37(b)N+Y39(b)S, L37(b)N+Y39(b)T, K40(b)N+P42(b)S, K40(b)N+P42(b)T, A43(b)N+P45(b)S, A43(b)N+P45(b)T, P45(b)N+I47(b)S, P45(b)N+I47(b)T, K46(b)N+Q48(b)S, K46(b)N+Q48(b)T, I47(b)N+K49(b)S, I47(b)N+K49(b)T, K54(b)N+L56(b)S, K54(b)N+L56(b)T, E55(b)N+V57(b)S, E55(b)N+V57(b)T, L56(b)N+Y58(b)S, L56(b)N+Y58(b)T, V57(b)N+E59(b)S, V57(b)N+E59(b)T, Y58(b)N+T60(b)S, Y58(b)N, E59(b)N+V61(b)S, E59(b)N+V61(b)T, T60(b)N+R62(b)S, T60(b)N+R62(b)T, R62(b)N+P64(b)S, R62(b)N+P64(b)T, G65(b)N+A67(b)S, G65(b)N+A67(b)T, A67(b)N+H69(b)S, A67(b)N+H69(b)T, H68(b)N+A70(b)S, H68(b)N+A70(b)T, H69(b)N+D71(b)S, H69(b)N+D71(b)T, D71(b)N+L73(b)S, D71(b)N+L73(b)T, L73(b)N+T75(b)S, L73(b)N, T75(b)N+P77(b)S, T75(b)N+P77(b)T, H83(b)N+G85(b)S, H83(b)N+G85(b)T, K86(b)N+D88(b)S, K86(b)N+D88(b)T, D88(b)N+D90(b)S, D88(b)N+D90(b)T, S89(b)N, S89(b)N+S91(b)T, D90(b)N+T92(b)S, D90(b)N, S91(b)N+D93(b)S, S91(b)N+D93(b)T, D93(b)N+T96(b)S, D93(b)N, T95(b)N+R97(b)S, T95(b)N+R97(b)T, V96(b)N+G98(b)S, V96(b)N+G98(b)T, R97(b)N+L99(b)S, R97(b)N+L99(b)T, L99(b)N+P101(b)S, L99(b)N+P101(b)T, Y103(b)N, Y103(b)N+S105(b)T, S105(b)N+G107(b)S, S105(b)N+G107(b)T, F106(b)N+E108(b)S, F106(b)N+E108(b)T, G107(b)N+M109(b)S, G107(b)N+M109(b)T, E108(b)N+K110(b)S, E108(b)N+K110(b)T, M109(b)N+E111(b)S, and M109(b)N+E111(b)T (mutations at positions with at least 25% side chain exposure). Preferably, glycosylation sites are introduced by means of mutation of a single amino acid residue selected from the group consisting of E4(b)N, Y58(b)N, L73(b)N, S89(b)N, D90(b)N, D93(b)N, and Y103(b)N.

More preferably, a modified FSH-β has an amino acid residue which differs from that of hFSH-β in the introduction of at least one N-glycosylation site by a mutation selected from the group consisting of F19(b)N+I21(b)S, F19(b)N+I21(b)T, Y33(b)N+R35(b)S, Y33(b)N+R35(b)T, A43(b)N+P45(b)S, A43(b)N+P45(b)T, P45(b)N+147(b)S, P45(b)N+I47(b)T, K46(b)N+Q48(b)S, K46(b)N+Q48(b)T, I47(b)N+K49(b)S, I47(b)N+K49(b)T, K54(b)N+L56(b)S, K54(b)N+L56(b)T, E55(b)N+V57(b)S, E55(b)N+V57(b)T, V57(b)N+E59(b)S, V57(b)N+E59(b)T, Y58(b)N+T60(b)S, Y58(b)N, E59(b)N+V61(b)S, E59(b)N+V61(b)T, R62(b)N+P64(b)S, R62(b)N+P64(b)T, G65(b)N+A67(b)S, G65(b)N+A67(b)T, A67(b)N+H69(b)S, A67(b)N+H69(b)T, H68(b)N+A70(b)S, H68(b)N+A70(b)T, H69(b)N+D71(b)S, H69(b)N+D71(b)T, D71(b)N+L73(b)S, D71(b)N+L73(b)T, L73(b)N+T75(b)S, L73(b)N, T75(b)N+P77(b)S, T75(b)N+P77(b)T, H83(b)N+G85(b)S, H83(b)N+G85(b)T, K86(b)N+D88(b)S, K86(b)N+D88(b)T, D88(b)N+D90(b)S, D88(b)N+D90(b)T, S89(b)N, S89(b)N+S91(b)T, D90(b)N+T92(b)S, D90(b)N, S91(b)N+D93(b)S, S91(b)N+D93(b)T, T95(b)N+R97(b)S, T95(b)N+R97(b)T, R97(b)N+L99(b)S, R97(b)N+L99(b)T, L99(b)N+P101(b)S, L99(b)N+P101(b)T, Y103(b)N, Y103(b)N+S105(b)T, S105(b)N+G107(b)S, S105(b)N+G107(b)T, F106(b)N+E108(b)S, F106(b)N+E108(b)T, G107(b)N+M109(b)S, G107(b)N+M109(b)T, E108(b)N+K110(b)S, E108(b)N+K110(b)T, M109(b)N+E111(b)S, and M109(b)N+E111(b)T (positions having more than 50% side chain accessibility). Among these positions, it is preferred to introduce glycosylation sites using mutation of a single amino acid residue selected from the group consisting of Y58(b)N, L73(b)N, S89(b)N, D90(b)N, and Y103(b)N.

The FSH-α part of such conjugates with an altered FSH-β subunit can be hFSH-α or any of the modified FSH-α polypeptides described herein.

The FSH-α and/or FSH-β polypeptide can further differ from hFSH-α and/or hFSH-β in at least one removed, naturally occurring N-glycosylation site. In particular, FSH-α can comprise a substitution of N78(a) and/or T80(a) by any other amino acid residue and/or FSH-β can comprise a substitution of N7(b), T9(b), N24(b) and/or T26(b) by any other amino acid residue. Preferably, the N residue is substituted by Q or D, and the T residue by A or G.

Furthermore, one or both of the FSH-α and FSH-β subunits of the conjugate according to this embodiment (having at least one of the above mentioned N-glycosylation site modifications) can differ from hFSH-α and hFSH-β, respectively, in the removal, preferably by substitution, of at least one lysine residue. See the section below on removal of lysine residues for further details.

An alternative embodiment of this aspect of the invention is one in which at least one of said FSH-α and FSH-β subunits comprises at least one introduced N- or O-glycosylation site at the N-terminal thereof, and wherein the at least one introduced glycosylation site is glycosylated; see the discussion of peptide addition below. In this case, the respective subunits can comprise one or more of the modifications disclosed elsewhere herein, or one or both of the subunits can be the respective wildtype subunits, but having the at least one introduced terminal glycosylation site. Thus, the polypeptide conjugate can be one in which the FSH-α subunit comprises hFSH-α having the sequence shown in SEQ ID NO:2, and/or in which the FSH-β subunit comprises hFSH-β having the sequence shown in SEQ ID NO:4. In a particular embodiment, both of the subunits correspond to the respective wildtype hFSH subunits, although with either the α or β subunit, or both, having an introduced N-terminal glycosylation site.

The introduced glycosylation site can be of the type described elsewhere herein; see the discussion of glycosylation under the general discussion of attachment groups above. A non-limiting example of a suitable glycosylation site for introduction at the N-terminal is the sequence Ala-Asn-Ile-Thr-Val-Asn-Ile-Thr-Val, e.g., for insertion of two glycosylation sites upstream of a mature FSH-α or FSH-β sequence.

Introduction of glycosylation sites by means of peptide addition

In addition to or as an alternative to introducing glycosylation sites within the amino acid sequence of one or both of the subunits, one or more additional glycosylation sites can be introduced by means of a “peptide addition” as discussed in the following. In this case, each of the polypeptide subunits comprises or consists of or consists essentially of the primary structure,

NH₂—X—P—COOH or NH₂—P—X—COOH,

wherein

X is a peptide addition comprising or contributing to a glycosylation site, and P is the basic polypeptide subunit to be modified, i.e., FSH-α or FSH-β, e.g., a wildtype polypeptide subunit as defined herein or a modified polypeptide having introduced and/or removed glycosylation sites or other attachment sites in the mature part of the polypeptide.

In the context of a peptide addition the term “comprising a glycosylation site” is intended to mean that a complete glycosylation site is present in the peptide addition, whereas the term “contributing to a glycosylation site” is intended to cover the situation where at least one amino acid residue of an N-glycosylation site is present in the peptide addition while the other amino acid residue of said site is present in the polypeptide P, whereby the glycosylation site can be considered to bridge the peptide addition and the polypeptide.

Usually, the peptide addition is fused to the N-terminal or C-terminal end of the polypeptide P as reflected in the above shown structure so as to provide an N- or C-terminal elongation of the polypeptide P, preferably at the N-terminal. However, it is also possible to insert the peptide addition within the amino acid sequence of the polypeptide P whereby the polypeptide comprises, consists of or consists essentially of the primary structure NH₂—Pₓ—X—P_{y}—COOH, wherein

Pₓ is an N-terminal part of the relevant polypeptide P,

P_{y} is a C-terminal part of said polypeptide P, and

X is a peptide addition comprising or contributing to a glycosylation site.

In order to minimize structural changes effected by the insertion of the peptide addition within the sequence of the polypeptide P, it is desirable that it be inserted in a non-structural part thereof. For instance, Pₓ can be a non-structural N-terminal part of a mature polypeptide P, and P_{y} a structural C-terminal part of said mature polypeptide, or Pₓ can be a structural N-terminal part of a mature polypeptide P, and P_{y} a non-structural C-terminal part of said mature polypeptide.

The term “non-structural part” is intended to indicate a part of either the C- or N-terminal end of the folded polypeptide subunit that is outside the first structural element, such as an α-helix or a β-sheet structure. The non-structural part can easily be identified in a three-dimensional structure or model of the polypeptide. If no structure or model is available, a non-structural part typically comprises or consists of the first or last 1-20 amino acid residues, such as 1-10 amino acid residues of the amino acid sequence constituting the mature form of the polypeptide.

When the peptide addition comprises only few amino acid residues, e.g., 1-5, such as 1-3 amino acid residues, and in particular one amino acid residue, the peptide addition can be inserted into a loop structure of the polypeptide P and thereby elongate the loop.

In principle, the peptide addition X can be any stretch of amino acid residues ranging from a single amino acid residue to a mature protein. In the present context, it is contemplated that each peptide addition will normally comprise up to about 50 amino acid residues, such as 2-30 or 3-20 amino acid residues. The peptide addition can be designed by a site-specific or random approach. In order to minimize the risk of an immunogenic response, however, it is preferable to select N- or C-terminal extensions of the FSH sequence that comprise peptide sequences that are part of naturally occurring human proteins. Non-limiting examples of such peptide sequences include the sequence NSTQNATA, which corresponds to positions 231 to 238 of the human calcium activated channel 2 precursor (to add two N-glycosylation sites to FSH), or the sequence ANLTVRNLTRNVTV, which corresponds to positions 538 to 551 of the human G protein coupled receptor 64 (to add three N-glycosylation sites to FSH).

Typically, each peptide addition X comprises 1-10 glycosylation sites. The peptide addition X can thus comprise 1, 2, 3, 4, 5, 6, 7, 8, 9 or 10 glycosylation sites. It is well known that a frequently occurring consequence of modifying an amino acid sequence of, e.g., a human protein is that new epitopes are created by such modification. Non-polypeptide moieties can be used to shield any new epitopes created by the peptide addition, and therefore it is desirable that sufficient glycosylation sites (or attachment groups for another non-polypeptide moiety, e.g., a polymer such as PEG) are present to enable shielding of all epitopes introduced into the sequence. This is e.g., achieved when the peptide addition X comprises at least one glycosylation site within a stretch of 30 contiguous amino acid residues, preferably as at least one glycosylation sites within 20 amino acid residues, more preferably at least one attachment group within 10 amino acid residues, in particular 1-3 attachment groups within a stretch of 10 contiguous amino acid residues in the peptide addition X.

Preferably, the glycosylation site of the peptide addition is an in vivo glycosylation site, preferably an N-glycosylation site. For instance, the peptide addition X can have the structure X₁-N-X₂-T/S/C-Z, wherein X₁ is a peptide comprising at least one amino acid residue or is absent, X₂ is any amino acid residue different from P, and Z is absent or is a peptide comprising at least one amino acid residue. For instance, X₁ can absent, X₂ can be an amino acid residue selected from the group consisting of I, A, G, V and S (all relatively small amino acid residues), and Z can comprise at least 1 amino acid residue. Z can e.g., be a peptide comprising up to 50 amino acid residues and e.g., up to 10 glycosylation sites.

Alternatively, X₁ can comprise at least one amino acid residue, e.g., 1-50 amino acid residues with 1-10 glycosylation sites, X₂ can be an amino acid residue selected from the group consisting of I, A, G, V and S, and Z can be absent.

Examples of peptide additions for use in the present invention are ANITVNITV, NDTVNFT and NITVNITV; see Examples 9 and 10 below, which illustrate addition of these sequences at the N-terminal of the FSH-α and β subunits.

The peptide addition can comprise one or more of these peptide sequences, i.e., at least two of said sequences either directly linked together or separated by one or more amino acid residues, or can contain two or more copies of any of these peptide sequence. It will be understood that the above specific sequences are given for illustrative purposes and thus do not constitute an exhaustive list of peptide sequences of use in the present invention.

In one embodiment, the peptide addition X has an N residue in position −2 or −1, and the polypeptide P or Pₓ has a T or an S residue in position +1 or +2, respectively, the residue numbering being made relative to the N-terminal amino acid residue of P or Pₓ, whereby an N-glycosylation site is formed. For instance, the polypeptide can have a T or S residue in position 2, preferably a T residue, and the peptide addition is AN or comprises AN as the C-terminal amino acid residues.

O-glycosylation

As an alternative or in addition to the mutations discussed above, the heterodimeric polypeptide can comprise one or more introduced O-glycosylation sites, for example the amino acid sequence AATPAP, which has been found to be an efficient signal sequence for O-glycosylation in vivo (Asada et al. (1999) Glycoconj. J. 16(7):321-6). The AATPAP sequence for O-glycosylation is preferably introduced by way of insertion at the N- and/or C-terminus of the FSH-α and/or FSH-β subunit.

Preparation of glycosylated conjugates

It will be understood that in order to prepare a conjugate according to this aspect, the polypeptide must be expressed in a glycosylating host cell capable of attaching oligosaccharide moieties at the glycosylation site(s) in vivo or alternatively subjected to in vitro glycosylation. Examples of glycosylating host cells are given in the section further below entitled “Coupling to an oligosaccharide moiety.”

In addition to an oligosaccharide moiety, the conjugate according to the aspect of the invention described in the present section can contain additional non-polypeptide moieties different from O-linked or N-linked oligosaccharide moieties, in particular a polymer molecule such as PEG as described herein conjugated to one or more attachment groups present in the polypeptide part of the conjugate. This is particularly relevant when a lysine residue (or any other amino acid residue comprising an attachment group for the polymer molecule in question) has been introduced and/or removed.

It will be understood that any of the amino acid changes specified in this section can be combined with any of the amino acid changes specified in the other sections herein disclosing specific amino acid changes.

Conjugate of the Invention wherein the Non-polypeptide Moiety is Attached to a Lysine or the N-terminal Amino Acid Residue

In a further preferred embodiment, the conjugate of the invention is one wherein the amino acid residue comprising an attachment group for the non-polypeptide moiety is a lysine residue and the non-polypeptide moiety is any molecule which has lysine as an attachment group. For instance, the non-polypeptide moiety can be a polymer molecule, in particular any of the molecules mentioned in the section entitled “Conjugation to a polymer molecule,” and preferably selected from the group consisting of linear or branched polyethylene glycol and polyalkylene oxide. Most preferably, the polymer molecule is mPEG-SPA or oxycarbonyl-oxy-N-dicarboxyimide PEG (U.S. Pat. No. 5,122,614).

The FSH-α and/or FSH-β having introduced and/or removed at least one lysine can advantageously be in vivo glycosylated, e.g., using naturally occurring glycosylation sites present in the relevant FSH polypeptide. However, in a particular embodiment, the conjugate is one wherein the amino acid sequence of FSH-α and/or FSH β differs from that of FSH-α and/or FSH-β in that an N-glycosylation site has been introduced and/or removed. Such introduced/removed sites can be any of those described in the section entitled “Conjugate of the invention wherein the non-polypeptide moiety is an oligosaccharide moiety.”

i) Removal of lysine residues

hFSH-α contains 6 lysine residues and hFSH-β 7. In order to avoid conjugation to one or more of these lysine residues, e.g., lysine residues located at or close to the receptor-binding site of hFSH, it can be desirable to remove at least one lysine residue. Accordingly, in one embodiment, the conjugate of the invention is one which comprises a modified FSH-α having an amino acid residue which differs from that of hFSH-α in the removal of at least one lysine residue selected from the group consisting of K44(a), K45(a), K51(a), K63(a), K75(a), and K91(a), in particular at least one amino acid residue selected from of the group consisting of K44(a), K45(a), K63(a), K75(a), and K91(a) (these residues having more than 25% of their side chain exposed to the surface), and preferably from the group consisting of K45(a), K63(a), K75(a), and K91(a) (these residues having more than 50% of their side chain exposed to the surface). The FSH-β part of this conjugate can be hFSH-β or any of the modified FSH-β polypeptides described herein.

In another embodiment, the conjugate of the invention is one which comprises a modified FSH-β having an amino acid residue which differs from that of hFSH-β in the removal of at least one lysine residue selected from the group consisting of K14(b), K40(b), K46(b), K49(b), K54(b), K86(b), and K110(b), in particular at least one amino acid residue selected from of the group consisting of K14(b), K40(b), K46(b), K49(b), K54(b), K86(b), and K110(b) (these residues having more than 25% of their side chain exposed to the surface), and preferably from the group consisting of K46(b), K54(b), K86(b), and K110(b) (these residues having more than 50% of their side chain exposed to the surface). The FSH-α part of this conjugate can be hFSH-α or any of the modified FSH-α polypeptides described herein.

In a further embodiment, the conjugate of the invention is one which comprises a modified FSH-α and a modified FSH-β, each of which differ from the corresponding hFSH subunit in the removal of at least one of the above identified lysine residues. For instance, the conjugate of the invention can be one wherein the modified FSH-α and modified FSH-β subunit differ from the corresponding hFSH subunit in at least one of K45(a), K63(a), K75(a), and K91(a) and at least one of K46(b), K54(b), K86(b), and K101(b).

The removal of any of the above lysine residues is preferably achieved by substitution by any other amino acid residue, in particular by an arginine or a glutamine residue.

ii) Introduction of lysine residues

In order to obtain a more extensive conjugation it can be desirable to introduce at least one non-naturally occurring lysine residue in hFSH, in particular in a position occupied by an amino acid residue having a side chain which is more than 25% surface exposed and which is not part of a cystine or located at a receptor binding site.

Accordingly, in a further embodiment, the conjugate of the invention is one which comprises a modified FSH-α having an amino acid residue which differs from that of hFSH-α in the introduction of at least one lysine residue in a position selected from the group consisting of A1(a), P2(a), D3(a), V4(a), Q5(a), D6(a), P8(a), E9(a), T11(a), L12(a), Q13(a), E14(a), P16(a), F17(a), Q20(a), P21(a), G22(a), A23(a), P24(a), L26(a), M29(a), F33(a), R42(a), S43(a), T46(a), L48(a), V49(a), Q50(a), N52(a), V61(a), S64(a), Y65(a), N66(a), R67(a), V68(a), T69(a), M71(a), G72(a), G73(a), F74(a), N78(a), T80(a), A81(a), H83(a), S85(a), T86(a), Y88(a), Y89(a), H90(a), and S92(a), in particular selected from of the group consisting of A1(a), P2(a), D3(a), V4(a), Q5(a), D6(a), P8(a), E9(a), T11(a), Q13(a), E14(a), P16(a), F17(a), Q20(a), P21(a), G22(a), A23(a), T46(a), L48(a), V49(a), Q50(a), N52(a), S64(a), N66(a), R67(a), T69(a), G72(a), G73(a), T86(a), Y89(a), H90(a), and S92(a) (these residues having more than 50% of their side chain exposed to the surface), and most preferably in the position R42(a) and/or R67(a), such as R67(a). The FSH-β part of this conjugate can be hFSH-β or any of the modified FSH-β polypeptides described herein.

In a further embodiment, the conjugate of the invention is one which comprises a modified FSH-β having an amino acid residue which differs from that of hFSH-β in the introduction of at least one lysine residue in a position selected from the group consisting of N1(b), S2(b), E4(b), L5(b), T6(b), N7(b), I8(b), T9(b), E15(b), E16(b), R18(b), F19(b), I21(b), S22(b), N24(b), Y31(b), Y33(b), R35(b), D36(b), L37(b), Y39(b), D41(b), P42(b), A43(b), R44(b), P45(b), I47(b), E55(b), L56(b), V57(b), Y58(b), E59(b), T60(b), V61(b), R62(b), P64(b), G65(b), A67(b), H68(b), H69(b), D71(b), L73(b), Y74(b), T75(b), T80(b), Q81(b), H83(b), G85(b), D88(b), S89(b), D90(b), S91(b), D93(b), T95(b), V96(b), R97(b), G98(b), L99(b), G100(b), Y103(b), S105(b), F106(b), G107(b), E108(b), M109(b), and E111(b), in particular selected from of the group consisting of N1(b), N7(b), T9(b), E15(b), E16(b), R18(b), F19(b), N24(b), Y33(b), D41(b), P42(b), A43(b), R44(b), P45(b), I47(b), E55(b), V57(b), Y58(b), E59(b), R62(b), P64(b), G65(b), A67(b), H68(b), H69(b), D71(b), L73(b), T75(b), Q81(b), H83(b), D88(b), S89(b), D90(b), S91(b), T95(b), R97(b), G98(b), L99(b), G100(b), Y103(b), S105(b), F106(b), G107(b), E108(b), M109(b), and E111(b) (these residues having more than 50% of their side chain exposed to the surface), and most preferably selected from the group consisting of R18(b), R35(b), R44(b), R62(b), and R97(b), such R18(b), R44(b), R62(b), and R97(b). The FSH-α part of this conjugate can be hFSH-α or any of the modified FSH-α polypeptides described herein.

In a further embodiment, the conjugate of the invention is one which comprises a modified FSH-α and a modified FSH-β, each of which differ from the corresponding hFSH subunit in the introduction of a lysine residue, preferably by substitution, in at least one of the above identified positions. For instance, the conjugate of the invention can be one wherein the modified FSH-α and modified FSH-β subunit differ from the corresponding hFSH subunit in that a lysine residue has been introduced in at least one of R42(a) and R67(a), and at least one of R18(b), R35(b), R44(b), R62(b), and R97(b), and more preferably in R67(a), and at least one of R18(b), R44(b), R62(b), R97(b).

iii) Introduction and removal of lysine residues

The conjugate of the invention can comprise at least one introduced lysine residue, in particular any of those described in the section entitled “Introduction of lysine residues,” and at least one removed lysine residue, in particular any of those described in the section entitled “Removal of lysine residues.”

Preferably, the conjugate comprises a modified FSH-α and/or a modified FSH-β which differs from the corresponding hFSH-α/β in at least one introduced and at least one removed lysine residue, wherein the lysine residue is introduced by substitution of an amino acid residue selected from the group consisting of R42(a) and R67(a), R18(b), R35(b), R44(b), R62(b), and R97(b), and more preferably from the group consisting of R67(a), R18(b), R44(b), R62(b), and R97(b) and removal of a lysine residue selected from the group consisting of K45(a), K63(a), K75(a), K91(a) K46(b), K54(b), K86(b), and K110(b), the removal preferably being achieved by substitution by any other amino acid residue, in particular by an arginine residue.

N-terminal Pegylation of FSH

As indicated above, one aspect of the invention relates to a polypeptide conjugate wherein at least one of the FSH-α and FSH-β subunits comprises a polymer molecule bound to the N-terminal thereof. Preferably, the polymer is a polyethylene glycol (PEG) such as mPEG; see the general discussion below regarding conjugates comprising polyethylene glycol-derived polymers.

In the case of N-terminal PEGylated FSH conjugates according to the invention, the respective subunits can comprise one or more of the modifications disclosed elsewhere herein, or one or both of the subunits can be the respective wildtype subunits with a PEG-derived polymer being attached at the N-terminal. Thus, the polypeptide conjugate can be one in which the FSH-α subunit comprises hFSH-α having the sequence shown in SEQ ID NO:2, and/or in which the FSH-β subunit comprises hFSH-β having the sequence shown in SEQ ID NO:4. In one embodiment, both of the subunits correspond to the respective wildtype hFSH subunits, although with either the α or β subunit, or both, being N-terminally PEGylated. In a preferred embodiment, however, at least one glycosylation site has been introduced into one or both of the subunits as described in detail above. In cases where at least one of the subunits has an N-terminally attached PEG molecule, it will often be desirable that no other PEG molecules are attached, e.g., to a lysine residue. In such cases, the polypeptide conjugate will thus comprise either one or two N-terminally attached PEG molecules as the sole polymer molecule(s).

Aldehyde-activated PEG and reduction using NaBH₃CN have been used to selectively pegylate the N-terminal α-amino group of proteins (see for instance U.S. Pat. No. 5,824,784 regarding N-terminal PEGylation of G-CSF). The N-terminus of the α and/or the β chain of wildtype FSH or a modified form of FSH can be PEGylated using similar methods. Reaction materials include purified FSH or a modified form of FSH, methoxy PEG aldehyde (M PEG CHO), and NaBH₃CN. In order to optimise yield, one can for instance vary: molar ratio of FSH, M-PEG-CHO and NaBH₃CN, time for establishment of the Schiff's base equilibrium (reaction between FSH and M-PEG-CHO before addition of NaBH₃CN), reaction time after addition of NaBH₃CN, temperature, pH, or reaction volume. The yield of PEGylated FSH forms can be analysed using Western blotting, mass spectrometry and N-terminal sequencing. In order to restrict PEGylation to only one of the two N-termini in FSH, PEGylation of the α or β chain can be selectively prevented by addition of a glutamine to the N-terminus. Spontaneous cyclisation of such an N-terminal glutamine residue will render it unaccessible for PEGylation. Such a glutamine residue can subsequently be removed using a pyroglutamyl aminopeptidase (for instance EC 3.4.19.3).

Conjugate of the Invention Having a Non-lysine Residue as an Attachment Group

Based on the present disclosure the skilled person will be aware that amino acid residues comprising other attachment groups can be introduced into and/or removed from FSH-α and/or FSH-β, using the same approach as that illustrated above by lysine residues. For instance, one or more amino acid residues comprising an acid group (glutamic acid or aspartic acid), asparagine, tyrosine or cysteine can be introduced into positions which in hFSH are occupied by amino acid residues having surface exposed side chains (i.e., the positions mentioned above as being of interest for introduction of lysine residues), or removed. As described above, introduction or removal of such amino acid residues is preferably performed by substitution. Preferably, Asp is substituted by Asn, Glu by Gln, Tyr by Phe, and Cys by Ser. Another possibility is introduction and/or removal of a histidine, e.g., by substitution with arginine.

Non-polypeptide Moiety of the Conjugate of the Invention

As indicated above, the non-polypeptide moiety of the conjugate of the invention is preferably selected from the group consisting of a polymer molecule, a lipophilic compound, an oligosaccharide moiety (by way of in vivo glycosylation) and an organic derivatizing agent. All of these agents can confer desirable properties to the polypeptide part of the conjugate, in particular an increased functional in vivo half-life and/or an increased serum half-life. The polypeptide part of the conjugate is often conjugated to only one type of non-polypeptide moiety, but can also be conjugated to two or more different types of non-polypeptide moieties, e.g., to a polymer molecule and an oligosaccharide moiety, to a lipophilic group and an oligosaccharide moiety, to an organic derivatizing agent and an oligosaccharide moiety, to a lipophilic group and a polymer molecule, etc. The conjugation to two or more different non-polypeptide moieties can be done simultaneously or sequentially. In a preferred embodiment of a polypeptide conjugated to different types of non-polypeptide moieties, the polypeptide is conjugated to one or more oligosaccharide moieties by in vivo glycosylation, and to one or more polymer molecules, preferably PEG, more preferably at an N-terminal, by conjugation in vitro.

Polypeptide of the Invention

In a further aspect, the invention relates to a modified FSH-α or a modified FSH-β polypeptide constituting part of a conjugate of the invention. The modified FSH-α and FSH-β are preferably glycosylated and thus further comprise N-linked and/or O-linked oligosaccharide moieties. Specific modified FSH-α and FSH-β polypeptides of the invention are those described in the section entitled “Conjugate of the invention.”

Methods of Preparing a Conjugate of the Invention

In the following sections “Conjugation to an oligosaccharide moiety,” “Conjugation to a polymer molecule,” “Conjugation to a lipophilic compound” and “Conjugation to an organic derivatizing agent,” conjugation to specific types of non-polypeptide moieties is described.

Coupling to an oligosaccharide moiety

For in vivo glycolyslation, conjugation to an oligosaccharide moiety takes place by means of a glycosylating, eucaryotic expression host. The expression host cell can be selected from fungal (filamentous fungal or yeast), insect or animal cells or from transgenic plant cells. In one embodiment, the host cell is a mammalian cell, such as a CHO cell, e.g., CHO K1, a BHK or HEK cell, e.g., HEK 293, an insect cell such as an SF9 cell, or a yeast cell, e.g., S. cerevisiae or Pichia pastoris, or any of the host cells mentioned hereinafter. Preferred cells for expression of an in vivo glycosylated protein of the invention are mammalian cells, in particular CHO cells.

Conjugation to a polymer molecule

The polymer molecule to be coupled to the polypeptide can be any suitable polymer molecule, such as a natural or synthetic homo-polymer or hetero-polymer, typically with a molecular weight in the range of 300-50,000 Da, such as 500-20,000 Da, more preferably in the range of 1000-15,000 Da, such as in the range of 1000-12,000 Da or 2000-10,000 Da. Examples of homo-polymers include a polyol (i.e., poly-OH), a polyamine (i.e., poly-NH₂) and a polycarboxylic acid (i.e., poly-COOH). A hetero-polymer is a polymer which comprises different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG is the preferred polymer molecule, since it has only few reactive groups capable of cross-linking compared to e.g., polysaccharides such as dextran. In particular, monofunctional PEG, e.g., methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control.

To effect covalent attachment of the polymer molecule(s) to the polypeptide, the hydroxyl end groups of the polymer molecule must be provided in activated form, i.e., with reactive functional groups. Suitable activated polymer molecules are commercially available, e.g., from Shearwater Polymers, Inc., Huntsville, Ala., USA, or from PolyMASC Pharmaceuticals plc, UK. Alternatively, the polymer molecules can be activated by conventional methods known in the art, e.g., as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and Pharmaceuticals, Polyethylene Glycol and Derivatives, incorporated herein by reference). Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. Nos. 5,932,462 and 5,643,575, both of which are incorporated herein by reference. Furthermore, the following publications, incorporated herein by reference, disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503 and EP 154 316.

The conjugation of the polypeptide and the activated polymer molecules is conducted by use of any conventional method, e.g., as described in the following references (which also describe suitable methods for activation of polymer molecules): R. F. Taylor, (1991), “Protein immobilisation. Fundamental and applications,” Marcel Dekker, N. Y.; S. S. Wong, (1992), “Chemistry of Protein Conjugation and Crosslinking,” CRC Press, Boca Raton; G. T. Hermanson et al., (1993), “Immobilized Affinity Ligand Techniques,” Academic Press, N.Y.). The skilled person will be aware that the activation method and/or conjugation chemistry to be used depends on the attachment group(s) of the polypeptide (examples of which are given further above), as well as the functional groups of the polymer (e.g., being amine, hydroxyl, carboxyl, aldehyde, sulfydryl, succinimidyl, maleimide, vinysulfone or haloacetate). The PEGylation can be directed towards conjugation to all available attachment groups on the polypeptide (i.e., such attachment groups that are exposed at the surface of the polypeptide) or can be directed towards one or more specific attachment groups, e.g., the N-terminal amino group (U.S. Pat. No. 5,985,265). Furthermore, the conjugation can be achieved in one step or in a stepwise manner (e.g., as described in WO 99/55377).

It will be understood that the PEGylation is designed so as to produce the optimal molecule with respect to the number of PEG molecules attached, the size and form of such molecules (e.g., whether they are linear or branched), and where in the polypeptide such molecules are attached. The molecular weight of the polymer to be used will be chosen taking into consideration the desired effect to be achieved. For instance, if the primary purpose of the conjugation is to achieve a conjugate having a high molecular weight and larger size (e.g., to reduce renal clearance), one can choose to conjugate either one or a few high molecular weight polymer molecules or a number of polymer molecules with a smaller molecular weight to obtain the desired effect. For epitope shielding, a sufficiently high number (e.g., 2-8, such as 3-6) of low molecular weight polymer molecules (e.g., with a molecular weight of about 5,000 Da) can be used to effectively shield all or most epitopes of the polypeptide.

When the protein is conjugated to only a single polymer molecule, for example where an N-terminal PEG molecule is the only polymer molecule, it will often be advantageous that the polymer molecule, which can be linear or branched, has a relatively high molecular weight, e.g., about 12-20 kDa.

In a specific embodiment, the polypeptide conjugate of the invention comprises a PEG molecule attached to most or substantially all of the lysine residues in the polypeptide available for PEGylation, in particular a linear or branched PEG molecule, e.g., with a molecular weight of about 5 kDa. In this case, it will normally be desirable to remove one or more of the lysines present in wildtype hFSH-α or hFSH-β in order to provide a more limited number of attachment sites and obtain a desired distribution of the PEG molecules. The polypeptide conjugate can further comprise a PEG molecule attached to the N-terminal amino acid residue in addition to the lysine residues.

Normally, the polymer conjugation is performed under conditions aiming at reacting as many of the available polymer attachment groups as possible with polymer molecules. This is achieved by means of a suitable molar excess of the polymer in relation to the polypeptide. Typical molar ratios of activated polymer molecules to polypeptide are up to about 1000-1, such as up to about 200-1 or up to about 100-1. In some cases, the ratio can be somewhat lower, however, such as up to about 50-1, 10-1 or 5-1.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. 24, 375-378.

Subsequent to the conjugation residual activated polymer molecules are blocked according to methods known in the art, e.g., by addition of primary amine to the reaction mixture, and the resulting inactivated polymer molecules removed by a suitable method.

Covalent in vitro coupling of carbohydrate moieties glycosides (such as dextran) to amino acid residues of the polypeptide can also be used, e.g., as described in WO 87/05330 and in Aplin et al., CRC Crit Rev. Biochem., pp. 259-306, 1981. The in vitro coupling of carbohydrate moieties or PEG to protein- and peptide-bound Gln-residues can be carried out by transglutaminases (TGases). Transglutaminases catalyse the transfer of donor amine groups to protein- and peptide-bound Gln residues in a so-called cross-linking reaction. The donor amine groups can be protein- or peptide-bound e.g., as the ε-amino group in Lys residues or can be part of a small or large organic molecule. An example of a small organic molecule functioning as amino-donor in TGase-catalysed cross-linking is putrescine (1,4-diaminobutane). An example of a larger organic molecule functioning as amino-donor in TGase-catalysed cross-linking is an amine-containing PEG (Sato et al., Biochemistry 35, 13072-13080).

TGases, in general, are highly specific enzymes, and not every Gln residue exposed on the surface of a protein is accessible to TGase-catalysed cross-linking to amino-containing substances. On the contrary, only a few Gln residues function naturally as TGase substrates but the exact parameters governing which Gln residues are good TGase substrates remain unknown. Thus, in order to render a protein susceptible to TGase-catalysed cross-linking reactions it is often a prerequisite at convenient positions to add stretches of amino acid sequence known to function very well as TGase substrates. Several amino acid sequences are known to be or to contain excellent natural TGase substrates e.g., substance P, elafin, fibrinogen, fibronectin, α₂-plasmin inhibitor, α-caseins, and β-caseins.

Conjugation to a lipophilic compound

The polypeptide and the lipophilic compound can be conjugated to each other either directly or by use of a linker. The lipophilic compound can be a natural compound such as a saturated or unsaturated fatty acid, a fatty acid diketone, a terpene, a prostaglandin, a vitamin, a carotenoid or steroid, or a synthetic compound such as a carbon acid, an alcohol, an amine and sulphonic acid with one or more alkyl, aryl, alkenyl or other multiple unsaturated compounds. The conjugation between the polypeptide and the lipophilic compound, optionally through a linker, can be done according to methods known in the art, e.g., as described by Bodanszky in Peptide Synthesis, John Wiley, New York, 1976 and in WO 96/12505.

Coupling to an organic derivatizing agent

Covalent modification of the polypeptide exhibiting FSH activity can be performed by reacting one or more attachment groups of the polypeptide with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonateat, pH 5.5-7.0, because this agent is relatively specific for the histidyl side chain. Para-bromophenacyl bromide is also useful. The reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourca, 2,4-pentanedione and transaminase-catalyzed reaction with glyoxylate. Arginyl residues are modified by reaction with one or several conventional reagents, among them phenylglyoxal, 2,3-butanedione, 1,2-cyclohexanedione, and ninhydrin. Derivatization of arginine residues requires that the reaction be performed under alkaline conditions because of the high pKa of the guanidine functional group.

Furthermore, these reagents can react with the groups of lysine as well as the arginine guanidino group. Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethyl-pentyl) carbodiimide. Furthermore, aspartyl and glutamyl residues are converted to asparaginyl and glutaminyl residues by reaction with ammonium ions.

Blocking of a functional site

It has been reported that excessive polymer conjugation can lead to a loss of activity of the polypeptide to which the polymer is conjugated. This problem can be eliminated by e.g., removal of attachment groups located at the functional site or by blocking the functional site prior to conjugation. The latter strategy constitutes a further embodiment of the invention (the first strategy being exemplified further above, e.g., by removal of lysine residues which can be located close to the functional site). More specifically, according to the second strategy the conjugation between the polypeptide and the non-polypeptide moiety is conducted under conditions where the functional site of the polypeptide is blocked by a helper molecule capable of binding to the functional site of the polypeptide.

Preferably, the helper molecule is one which specifically recognizes a functional site of the polypeptide, such as a receptor, in particular the FSH receptor or a part of the FSH receptor. Alternatively, the helper molecule can be an antibody, in particular a monoclonal antibody recognizing the polypeptide exhibiting FSH activity. In particular, the helper molecule can be a neutralizing monoclonal antibody.

The polypeptide is allowed to interact with the helper molecule before effecting conjugation. This ensures that the functional site of the polypeptide is shielded or protected and consequently unavailable for derivatization by the non-polypeptide moiety such as a polymer. Following its elution from the helper molecule, the conjugate between the non-polypeptide moiety and the polypeptide can be recovered with at least a partially preserved functional site.

The subsequent conjugation of the polypeptide having a blocked functional site to a polymer, a lipophilic compound, an oligosaccharide moiety, an organic derivatizing agent or any other compound is conducted in the normal way, e.g., as described in the sections above entitled “Conjugation to . . . ”

Irrespective of the nature of the helper molecule to be used to shield the functional site of the polypeptide from conjugation, it is desirable that the helper molecule is free of or comprises only a few attachment groups for the non-polypeptide moiety of choice in any parts of the molecule where the conjugation to such groups will hamper the desorption of the conjugated polypeptide from the helper molecule. Hereby, selective conjugation to attachment groups present in non-shielded parts of the polypeptide can be obtained and it is possible to reuse the helper molecule for repeated cycles of conjugation. For instance, if the non-polypeptide moiety is a polymer molecule such as PEG which has the epsilon amino group of a lysine or N-terminal amino acid residue as an attachment group, it is desirable that the helper molecule is substantially free of conjugatable epsilon amino groups, preferably free of any epsilon amino groups. Accordingly, in a preferred embodiment, the helper molecule is a protein or peptide capable of binding to the functional site of the polypeptide, which protein or peptide is free of any conjugatable attachment groups for the non-polypeptide moiety of choice.

In a further embodiment, the helper molecule is first covalently linked to a solid phase such as column packing materials, for instance Sephadex or agarose beads, or a surface, e.g., a reaction vessel. Subsequently, the polypeptide is loaded onto the column material carrying the helper molecule and conjugation carried out according to methods known in the art, e.g., as described in the sections above entitled “Conjugation to . . . ” This procedure allows the polypeptide conjugate to be separated from the helper molecule by elution. The polypeptide conjugate is eluated by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule can be derivatised with a second molecule (e.g., biotin) that can be recognized by a specific binder (e.g., streptavidin). The specific binder can be linked to a solid phase thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate can be released from the helper molecule in any appropriate fashion. Deprotection can be achieved by providing conditions in which the helper molecule dissociates from the functional site of the FSH to which it is bound. For instance, a complex between an antibody to which a polymer is conjugated and an anti-idiotypic antibody can be dissociated by adjusting the pH to an acid or alkaline pH.

Conjugation of a tagged polypeptide

In an alternative embodiment, the polypeptide is expressed as a fusion protein with a tag, i.e., an amino acid sequence or peptide stretch made up of typically 1-30, such as 1-20 amino acid residues. Besides allowing for fast and easy purification, the tag is a convenient tool for achieving conjugation between the tagged polypeptide and the non-polypeptide moiety. In particular, the tag can be used for achieving conjugation in microtiter plates or other carriers, such as paramagnetic beads, to which the tagged polypeptide can be immobilised via the tag. The conjugation to the tagged polypeptide in, e.g., microtiter plates has the advantage that the tagged polypeptide can be immobilised in the microtiter plates directly from the culture broth (in principle without any purification) and subjected to conjugation. Thereby, the total number of process steps (from expression to conjugation) can be reduced. Furthermore, the tag can function as a spacer molecule ensuring an improved accessibility to the immobilised polypeptide to be conjugated. The conjugation using a tagged polypeptide can be to any of the non-polypeptide moieties disclosed herein, e.g., to a polymer molecule such as PEG.

The identity of the specific tag to be used is not critical as long as the tag is capable of being expressed with the polypeptide and is capable of being immobilised on a suitable surface or carrier material. A number of suitable tags are commercially available, e.g., from Unizyme Laboratories, Denmark. For instance, the tag can consist of any of the following sequences:

His-His-His-His-His-His
Met-Lys-His-His-His-His-His-His
Met-Lys-His-His-Ala-His-His-Gln-His-His
Met-Lys-His-Gln-His-Gln-His-Gln-His-Gln-His-Gln-His-Gln
Met-Lys-His-Gln-His-Gln-His-Gln-His-Gln-His-Gln-His-Gln-Gln
or any of the following:
EQKLI SEEDL (a C-terminal tag described in Mol. Cell. Biol. 5:3610-16, 1985)
DYKDDDDK (a C- or N-terminal tag)
YPYDVPDYA

Antibodies against the above tags are commercially available, e.g., from ADI, Aves Lab and Research Diagnostics.

The subsequent cleavage of the tag from the polypeptide can be achieved by use of commercially available enzymes.

Methods for Preparing a Polypeptide of the Invention or the Polypeptide of the Conjugate of the Invention

The polypeptide of the present invention or the polypeptide part of a conjugate of the invention, optionally in glycosylated form, can be produced by any suitable method known in the art. Such methods include constructing a nucleotide sequence encoding the polypeptide and expressing the sequence in a suitable transformed or transfected host. Polypeptides of the invention can also be produced, albeit less efficiently, by chemical synthesis or a combination of chemical synthesis and recombinant DNA technology.

FSH-α and FSH-β are preferably expressed by the same host cell, thus becoming dimerized in vivo prior to purification and possible in vitro conjugation to a non-polypeptide moiety. Co-expression of FSH-α and FSH-β in CHO cells is e.g., described by Keene et al., J Biol Chem 1989 25; 264(9): 4769-75. Alternatively, the polypeptide can be expressed as a single-chain polypeptide wherein the nucleotide sequences encoding FSH-α and FSH-β are fused, either directly or using a suitable peptide linker, and expressed as a single-chain polypeptide using a similar approach to that described in U.S. Pat. No. 5,883,073 or WO 96/05224. It will thus be understood that the polypeptide of the invention can comprise the FSH-α and FSH-β subunits in the form of two separate polypeptide chains, where the two chains become dimerized in vivo so as to form a dimeric polypeptide, or it can comprise a single-chain construct comprising the two subunits covalently linked by a peptide bond or a peptide linker.

In an alternative embodiment, two FSH-β subunits, wherein at least one of the two β subunits is modified as described herein, preferably by introduction of at least one N- or O-glycosylation site, can be expressed as a single-chain polypeptide in which the subunits are either fused directly or via a peptide linker. Similarly, two FSH-α subunits, wherein at least one of the two α subunits is modified as described herein, can also be expressed as a single-chain polypeptide with the subunits fused directly or via a peptide linker. Further, it is also possible to produce single-chain constructs comprising more than two subunits, e.g., three subunits, wherein at least one of the individual subunits is modified as described herein, and wherein the subunits are fused to each other directly or via a peptide linker. For example, a single-chain construct having the sequence FSHα-FSHβ-FSHβ, FSHβ-FSHα-FSHβ or FSHβ-FSHβ-FSHα, wherein the β subunits in each construct are the identical or different, can be produced using techniques known in the art. Single-chain constructs of this general type are disclosed in U.S. Pat. Nos. 5,705,478, 5,883,073, WO 99/25489 and WO 96/05224

For single-chain constructs, the linker peptide will often predominantly include the amino acid residues Gly, Ser, Ala and/or Thr. Such a linker typically comprises 1-30 amino acid residues, such as a sequence of about 2-20 or 3-15 amino acid residues. The amino acid residues selected for inclusion in the linker peptide should exhibit properties that do not interfere significantly with the activity of the polypeptide. Thus, the linker peptide should on the whole not exhibit a charge which would be inconsistent with the desired FSH activity, or interfere with internal folding, or form bonds or other interactions with amino acid residues in one or more of the subunits which would seriously impede the binding of the dimeric or multimeric polypeptide to the receptor.

Specific linkers for use in the present invention can be designed on the basis of known naturally occurring as well as artificial polypeptide linkers (see, e.g., Hallewell et al. (1989), J. Biol. Chem. 264, 5260-5268; Alfthan et al. (1995), Protein Eng. 8, 725-731; Robinson & Sauer (1996), Biochemistry 35, 109-116; Khandekar et al. (1997), J. Biol. Chem. 272, 32190-32197; Fares et al. (1998), Endocrinology 139, 2459-2464; Smallshaw et al. (1999), Protein Eng. 12, 623-630; U.S. Pat. No. 5,856,456). For instance, linkers used for creating single-chain antibodies, e.g., a 15mer consisting of three repeats of a Gly-Gly-Gly-Gly-Ser amino acid sequence ((Gly₄Ser)₃), are contemplated to be useful. Furthermore, phage display technology as well as selective infective phage technology can be used to diversify and select appropriate linker sequences (Tang et al., J. Biol. Chem. 271, 15682-15686, 1996; Hennecke et al. (1998), Protein Eng. 11, 405-410). Also, Arc repressor phage display has been used to optimize the linker length and composition for increased stability of a single-chain protein (Robinson and Sauer (1998), Proc. Natl. Acad. Sci. USA 95, 5929-5934).

Another way of obtaining a suitable linker is by optimizing a simple linker, e.g., ((Gly₄Ser)ₙ), through random mutagenesis. The linker can e.g., be (Gly₄Ser)ₙ or (Gly₃Ser)ₙ where n is 1, 2, 3 or 4.

The nucleotide sequence encoding FSH-α or FSH-β modified according to the invention can be constructed by isolating or synthesizing a nucleotide sequence encoding the parent FSH subunit, such as hFSH-α or hFSH-β with the amino acid sequence shown in SEQ ID NO:2 or 4, respectively, or the precursor form thereof (shown in SEQ ID NO:1 and 3, respectively) and then changing the nucleotide sequence so as to effect introduction (i.e., insertion or substitution) or deletion (i.e., removal or substitution) of the relevant amino acid residue(s). The nucleotide sequence is conveniently modified by site-directed mutagenesis in accordance with conventional methods. Alternatively, the nucleotide sequence can be prepared by chemical synthesis, e.g., by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide can be synthesized and assembled by PCR, ligation or ligation chain reaction (LCR) (Barany, PNAS 88:189-193, 1991). The individual oligonucleotides typically contain 5′ or 3′ overhangs for complementary assembly.

Once assembled (by synthesis, site-directed mutagenesis or another method), the nucleotide sequence encoding the polypeptide is inserted into a recombinant vector and operably linked to control sequences necessary for expression of the FSH in the desired transformed host cell.

It should of course be understood that not all vectors and expression control sequences function equally well to express the nucleotide sequence encoding a polypeptide described herein. Neither will all hosts function equally well with the same expression system. However, one of skill in the art can make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and the ease of purification of the products coded for by the nucleotide sequence.

The recombinant vector can be an autonomously replicating vector, i.e., a vector which exists as an extrachromosomal entity, the replication of which is independent of chromosomal replication, e.g., a plasmid. Alternatively, the vector is one which, when introduced into a host cell, is integrated into the host cell genome and replicated together with the chromosome(s) into which it has been integrated.

The vector is preferably an expression vector in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for mammalian eukaryotic hosts include, for example, vectors comprising expression control sequences from SV40, bovine papilloma virus, adenovirus and cytomegalovirus. Specific vectors are, e.g., pCDNA3.1(+)\Hyg (Invitrogen, Carlsbad, Calif., USA) and pCI-neo (Stratagene, La Jolla, Calif., USA). Useful expression vectors for yeast cells include the 2μ plasmid and derivatives thereof, the POT1 vector (U.S. Pat. No. 4,931,373), the pJSO37 vector described in Okkels, Ann. New York Acad. Sci. 782, 202-207, 1996, and pPICZ A, B or C (Invitrogen). Useful vectors for insect cells include pVL941, pBG311 (Cate et al., Cell 45, pp. 685-98 (1986)), pBluebac 4.5 and pMelbac (both available from Invitrogen). Useful expression vectors for bacterial hosts include known bacterial plasmids, such as plasmids from E. coli, including pBR322, pET3a and pET12a (both from Novagen Inc., WI, USA), wider host range plasmids, such as RP4, phage DNAs, e.g., the numerous derivatives of phage lambda, e.g., NM989, and other DNA phages, such as M13 and filamentous single stranded DNA phages.

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, Mol. Cell. Biol. 2, pp. 1304-19 (1982)) and glutamine synthetase (“GS”) amplification (see, e.g., U.S. Pat. No. 5,122,464 and EP 338,841).

In one embodiment, a pair of expression vectors are used for expressing the polypeptide subunits of the invention. Each of the vectors of said pair is capable of transfecting a eukaryotic cell as described herein, and the vectors comprise nucleotide sequences encoding, respectively, a modified FSH-α as described herein and a wildtype FSH-β subunit, a modified FSH-β as described herein and a wildtype FSH-α subunit, or a modified FSH-α and a modified FSH-β as described herein. The use of a pair of vectors is e.g., described in EP 211,894. Alternatively, a single expression vector comprising nucleotide sequences encoding both the FSH-α subunit and the FSH-β subunit, where at least one of the subunits is modified as described herein, can be used for expressing the polypeptide subunits.

The recombinant vector can further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector can also comprise a selectable marker, e.g., a gene whose product complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g., ampicillin, kanamycin, tetracyclin, chloramphenicol, neomycin, hygromycin or methotrexate. For Saccharomyces cerevisiae, selectable markers include ura3 and leu2. For filamentous fungi, selectable markers include amdS, pyrG, arcB, niaD and sC.

The term “control sequences” is defined herein to include all components which are necessary or advantageous for the expression of the polypeptide of the invention. Each control sequence can be native or foreign to the nucleic acid sequence encoding the polypeptide. Such control sequences include, but are not limited to, a leader sequence, polyadenylation sequence, propeptide sequence, promoter, enhancer or upstream activating sequence, signal peptide sequence, and transcription terminator. At a minimum, the control sequences include a promoter.

A wide variety of expression control sequences can be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g., the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol Aug. 20, 1987;196(4):947-50).

In order to improve expression in mammalian cells a synthetic intron can be inserted in the 5′ untranslated region of the nucleotide sequence encoding the polypeptide. An example of a synthetic intron is the synthetic intron from the plasmid pCI-Neo (available from Promega Corporation, WI, USA).

Examples of suitable control sequences for directing transcription in insect cells include the polyhedrin promoter, the P10 promoter, the Autographa californica polyhedrosis virus basic protein promoter, the baculovirus immediate early gene 1 promoter and the baculovirus 39K delayed-early gene promoter, and the SV40 polyadenylation sequence. Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4c promoter, and the inducible GAL promoter. Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator. Examples of suitable control sequences for use in bacterial host cells include promoters of the lac system, the trp system, the TAC or TRC system, and the major promoter regions of phage lambda.

The presence or absence of a signal peptide will, e.g., depend on the expression host cell used for the production of the polypeptide to be expressed (whether it is an intracellular or extracellular polypeptide) and whether it is desirable to obtain secretion. For use in filamentous fungi, the signal peptide can conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide can conveniently be derived from an insect gene (cf. WO 90/05783), such as the Lepidopteran manduca sexta adipokinetic hormone precursor, (cf. U.S. Pat. No. 5,023,328), the honeybee melittin (Invitrogen), ecdysteroid UDPglucosyltransferase (egt) (Murphy et al., Protein Expression and Purification 4, 349-357 (1993) or human pancreatic lipase (hpl) (Methods in Enzymology 284, pp. 262-272, 1997). A preferred signal peptide for use in mammalian cells is that of hFSH or the murine Ig kappa light chain signal peptide (Coloma, M (1992) J. Imm. Methods 152:89-104). For use in yeast cells suitable signal peptides have been found to be the α-factor signal peptide from S. cereviciae (cf. U.S. Pat. No. 4,870,008), a modified carboxypeptidase signal peptide (cf. L. A. Valls et al., Cell 48, 1987, pp. 887-897), the yeast BAR1 signal peptide (cf. WO 87/02670), the yeast aspartic protease 3 (YAP3) signal peptide (cf. M. Egel-Mitani et al., Yeast 6, 1990, pp. 127-137), and the synthetic leader sequence TA57 (WO98/32867). For use in E. coli cells a suitable signal peptide have been found to be the signal peptide ompA (EP581821).

The nucleotide sequences of the invention encoding the dimeric polypeptide exhibiting FSH activity, whether prepared by site-directed mutagenesis, synthesis, PCR or other methods, can optionally also include a nucleotide sequence that encodes a signal peptide. The signal peptide is present when the polypeptide is to be secreted from the cells in which it is expressed. Such signal peptide, if present, should be one recognized by the cell chosen for expression of the polypeptide. The signal peptide can be homologous (e.g., be that normally associated with a hFSH subunit) or heterologous (i.e., originating from another source than hFSH) to the polypeptide or can be homologous or heterologous to the host cell, i.e., be a signal peptide normally expressed from the host cell or one which is not normally expressed from the host cell. Accordingly, the signal peptide can be prokaryotic, e.g., derived from a bacterium such as E. coli, or eukaryotic, e.g., derived from a mammalian, or insect or yeast cell.

Any suitable host can be used to produce the polypeptide subunits of the invention, including bacteria, fungi (including yeasts), plant, insect, mammal, or other appropriate animal cells or cell lines, as well as transgenic animals or plants. Examples of bacterial host cells include gram-positive bacteria such as strains of Bacillus, e.g., B. brevis or B. subtilis, or Streptomyces, or gram-negative bacteria, such as Pseudomonas or strains of E. coli. The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278). Examples of suitable filamentous fungal host cells include strains of Aspergillus, e.g., A. oryzae, A. niger, or A. nidulans, Fusarium or Trichodenna. Fungal cells can be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Examples of suitable yeast host cells include strains of Saccharomyces, e.g., S. cerevisiae, Schizosaccharomyces, Klyveromyces, Pichia, such as P. pastoris or P. methanolica, Hansenula, such as H. Polymorpha or Yarrowia. Yeast can be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; Hinnen et al., 1978, PNAS USA 75: 1920: and as disclosed by Clontech Laboratories, Inc, Palo Alto, Calif., USA (in the product protocol for the Yeastmaker™ Yeast Transformation System Kit). Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein can be performed as described by Invitrogen. Examples of suitable mammalian host cells include Chinese hamster ovary (CHO) cell lines, (e.g., CHO-K1; ATCC CCL-61), Green Monkey cell lines (COS) (e.g., COS 1 (ATCC CRL-1650), COS 7 (ATCC CRL-1651)); mouse cells (e.g., NS/O), Baby Hamster Kidney (BHK) cell lines (e.g., ATCC CRL-1632 or ATCC CCL-10), and human cells (e.g., HEK 293 (ATCC CRL-1573)), as well as plant cells in tissue culture. Additional suitable cell lines are known in the art and available from public depositories such as the American Type Culture Collection, USA. Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g., described by Ausbel et al. (eds.), 1996, Current Protocols in Molecular Biology, John Wiley & Sons, NY, USA. The cultivation of mammalian cells are conducted according to established methods, e.g., as disclosed in (Animal Cell Biotechnology, Methods and Protocols, Edited by Nigel Jenkins, 1999, Human Press Inc, Totowa, N.J., USA and Harrison M A and Rae I F, General Techniques of Cell Culture, Cambridge University Press 1997).

In the production methods of the present invention, the cells are cultivated in a nutrient medium suitable for production of the polypeptide using methods known in the art. For example, the cell can be cultivated by shake flask cultivation, small-scale or large-scale fermentation (including continuous, batch, fed-batch, or solid state fermentations) in laboratory or industrial fermenters performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection). If the polypeptide is secreted into the nutrient medium, it can be recovered directly from the medium. If the polypeptide is not secreted, it can be recovered from cell lysates.

The resulting polypeptide can be recovered by methods known in the art. For example, it can be recovered from the nutrient medium by conventional procedures including, but not limited to, centrifugation, filtration, extraction, spray drying, evaporation, or precipitation.

The polypeptides can be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see e.g., Protein Purification, J.-C. Janson and Lars Ryden, editors, VCH Publishers, New York, 1989).

Pharmaceutical Composition of the Invention and its use

In one aspect, the polypeptide, the conjugate or the pharmaceutical composition according to the invention is used for the manufacture of a medicament for treatment of infertility or diseases associated with insufficient endogenous production of FSH.

In another aspect, the polypeptide, the conjugate or the pharmaceutical composition according to the invention is used in a method of treating an infertile mammal, in particular a human, comprising administering to the mammal in need thereof such polypeptide, conjugate or pharmaceutical composition.

The polypeptide exhibiting FSH activity of the invention or the conjugate of the invention is administered at a dose approximately paralleling that employed in therapy with rhFSH such as Gonal-F® and Puregon®. However, due to the increased functional in vivo half-life of the conjugate of the invention, it is contemplated that the product will be administered less frequently and at a dose which provides a comparable effect to that obtained in current therapy. It is thus contemplated that the composition of the invention can be administered at substantially less frequent intervals than currently available treatments, e.g., not more often than once every three days, such as not more than once every four, five, six or seven days. Accordingly, the exact dose to be administered will depend on the circumstances, including the patient to be treated, the cause of infertility if known, the status of the ovaries, the patient's plasma FSH concentration prior to treatment, and the functional in vivo half-life of the product. Normally, in the treatment of infertility the dose should be capable of stimulating follicle maturation, e.g., induce follicles to grow about 2 mm per day during a time period of 8-9 days. For instance, for a product having a functional in vivo half-life of 3-4 days, two doses should be given at least three days apart if a relatively stable plasma concentration is desired. Analogously, for a product having a functional in vivo half-life of about 6 days, one dose would suffice during most of the stimulation period.

The composition of the invention can be exceedingly advantageous when employed in a step-down protocol, i.e., a protocol where decreasing dosages of FSH are given during the stimulation period, but where use of the composition of the invention, e.g., administered in one or two doses as outlined above, can provide such a slowly decreasing plasma concentration of FSH.

It will be apparent to those of skill in the art that an effective amount of a conjugate, preparation or composition of the invention depends, inter alia, upon the disease, the dose, the administration schedule, whether the polypeptide or conjugate or composition is administered alone or in conjunction with other therapeutic agents, the serum half-life of the compositions, and the general health of the patient. Typically, an effective dose of the conjugate, preparation or composition of the invention is sufficient to ensure development and maturation of follicles at a rate and to a degree compatible with that obtained using standard rhFSH such as Gonal-F® and Puregon®.

A further contemplated advantage is that the more stable plasma concentration obtained with a composition of the invention results in a more efficient development and maturation of follicles, which subsequently can enable a higher pregnancy rate.

The polypeptide or conjugate of the invention is normally administered in a composition including one or more pharmaceutically acceptable carriers or excipients. “Pharmaceutically acceptable” means a carrier or excipient that does not cause any untoward effects in patients to whom it is administered. Such pharmaceutically acceptable carriers and excipients are well known in the art, and the polypeptide or conjugate of the invention can be formulated into pharmaceutical compositions by well-known methods (see e.g., Remington's Pharmaceutical Sciences, 18th edition, A. R. Gennaro, Ed., Mack Publishing Company (1990); Pharmaceutical Formulation Development of Peptides and Proteins, S. Frokjaer and L. Hovgaard, Eds., Taylor & Francis (2000); and Handbook of Pharmaceutical Excipients, 3rd edition, A. Kibbe, Ed., Pharmaceutical Press (2000)). Pharmaceutically acceptable excipients that can be used in compositions comprising the polypeptide or conjugate of the invention include, for example, buffering agents, stabilizing agents, preservatives, isotonifiers, non-ionic surfactants or detergents (“wetting agents”), antioxidants, bulking agents or fillers, chelating agents and cosolvents.

The pharmaceutical composition of the polypeptide or conjugate of the invention can be formulated in a variety of forms, including liquids, e.g., ready-to-use solutions or suspensions, gels, lyophilized, or any other suitable form, e.g., powder or crystals suitable for preparing a solution. The preferred form will depend upon the particular indication being treated and will be apparent to one of skill in the art.

The pharmaceutical composition containing the polypeptide or conjugate of the invention can be administered intravenously, intramuscularly, intraperitoneally, intradermally, subcutaneously, sublingualy, buccally, intranasally, transdermally, by inhalation, or in any other acceptable manner, e.g., using PowderJect® or ProLease® technology or a pen injection system. The preferred mode of administration will depend upon the particular indication being treated and will be apparent to one of skill in the art. In particular, it is advantageous that the composition be administered subcutaneously, since this allows the patient to conduct the administration herself.

The pharmaceutical composition of the invention can be administered in conjunction with other therapeutic agents. These agents can be incorporated as part of the same pharmaceutical composition or can be administered separately from the polypeptide or conjugate of the invention, either concurrently or in accordance with any other acceptable treatment schedule. In addition, the polypeptide, conjugate or pharmaceutical composition of the invention can be used as an adjunct to other therapies.

By obtaining a more stable FSH plasma concentration just above the threshold level for follicle growth, the composition of the invention is of particular interest for the treatment of women suffering from anovulation WHO type I, II or III, since only 1-2 mature follicles are desired in these patients.

Furthermore, the invention relates in other aspects to the use of a composition of the invention in a step-down protocol where a decreasing plasma FSH concentration is obtained using only one or two injections, and preferably only a single injection, to the use of a composition of the invention in a step-up protocol where an increase in FSH concentrations is obtained faster using a lower individual as well as total dosage, and to the use of a composition of the invention in combination with compounds for in vitro maturation (sterol derivatives such as FF-MAS and media containing growth and maturation factors known in the art).

Mixtures of FSH and LH activities (hMG) are routinely used in the treatment of human infertility. This particular combination therapy can be advantageous because gonadal support of gamete maturation is dependent upon the synergistic actions of both FSH and LH. Current treatment protocols requiring FSH and LH activity utilize urinary extracts from postmenopausal women. The use of these extracts is compromised by several factors, including variability.

It will in some cases be advantageous to administer the composition of the invention as part of a treatment protocol that also involves LH and/or hCG, for example recombinant LH and/or hCG. This can in particular be useful for treatment of women with low endogenous LH levels. Finally, the composition of the invention can be used, possibly in combination with LH, in the treatment of male infertility, in particular of hypogonadotrophic hypogonadism and oligo- or azoospermia. The more stable plasma concentration obtained with a composition of the invention can lead to a more efficient spermatogenesis. Also, a long lasting effect would be particularly advantageous for such treatment due to the long-term treatment period of about three months.

The present invention will be further illustrated by the following non-limiting methods and examples.

Structure Analysis Methods

Sequence numbering

The amino acid sequence of hFSH-α is numbered according to the mature sequence shown in SEQ ID NO:2; an (a) suffix herein indicates the α chain. The amino acid sequence of hFSH-β is numbered according to the mature sequence shown in SEQ ID NO:4; a (b) suffix herein indicates the β chain.

Structures

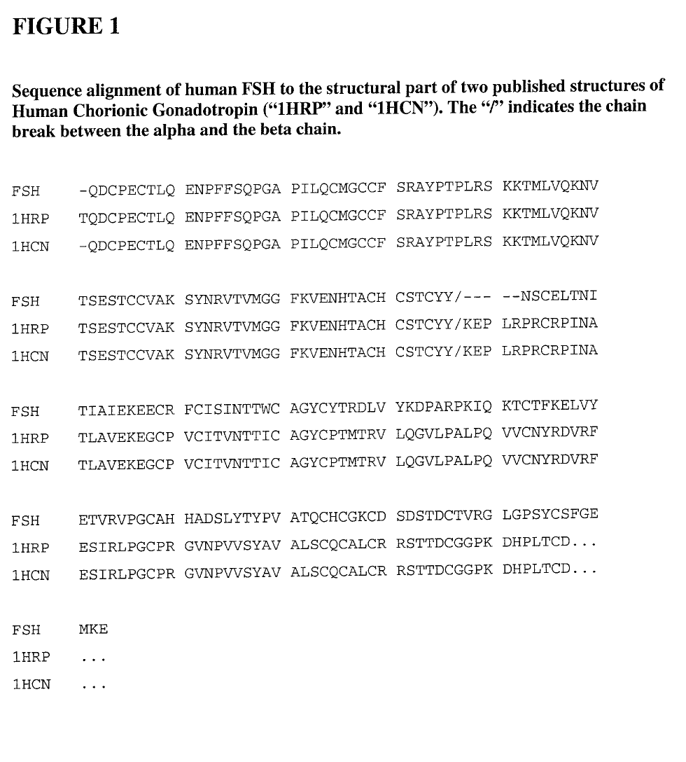

Human FSH α is identical to the α chain of Human Chorionic Gonadotropin (HCG) for which two published structures are available: Wu, H., Lustbader, J. W., Liu, Y., Canfield, R. E., Hendrickson, W. A.: Structure 2 pp. 545 (1994) and Lapthorn, A. J., Harris, D. C., Littlejohn, A., Lustbader, J. W., Canfield, R. E., Machin, K. J., Morgan, F. J., Isaacs, N. W.: Nature 369 pp. 455 (1994), both including the β chain of HCG. The β chain of hFSH is 32 percent identical to the amino acid sequence of the structural part of the β chain of HCG (see the sequence alignment of FIG. 1). A series of 50 models of the 3D structure of FSH was built based on the above two available hCG structures and based on the sequence alignment in FIG. 1 using the program Modeller 98 (MSI Inc., 1999). The four N-terminal residues (A1(a), P2(a), D3(a) and V4(a) as well as the three C-terminal residues (H90(a), K91(a) and S92(a) were not modeled as they are not identified in the HCG structures. All of the HFSH-β chain was modeled, even the part which has no homologous residues in the HCG structures.

Accessible Surface Area (ASA)

The computer program Access (B. Lee and F. M. Richards, J. Mol. Biol. 55: 379-400 (1971)) version 2 (®1983 Yale University) was used to compute the accessible surface area (ASA) of the individual atoms in the structure. This method typically uses a probe-size of 1.4 Å and defines the Accessible Surface Area (ASA) as the area formed by the center of the probe. Prior to this calculation all water molecules and all hydrogen atoms should be removed from the coordinate set, as should other atoms not directly related to the protein.

Fractional ASA of side chain

The fractional ASA of the side chain atoms is computed by division of the sum of the ASA of the atoms in the side chain with a value representing the ASA of the side chain atoms of that residue type in an extended Ala-x-Ala tripeptide, see Hubbard, Campbell & Thornton (1991) J. Mol. Biol. 220,507-530. For this example the CA atom is regarded as being a part of the side chain of glycine residues but not other residues. The following values are used as standard 100% ASA for the side chain:

| | | | | | |
|---|---|---|---|---|---|
| Ala | 69.23 | Å² | Leu | 140.76 | Å² |
| Arg | 200.35 | Å² | Lys | 162.50 | Å² |
| Asn | 106.25 | Å² | Met | 156.08 | Å² |
| Asp | 102.06 | Å² | Phe | 163.90 | Å²^{¹⁵} |
| Cys | 96.69 | Å² | Pro | 119.65 | Å² |
| Gln | 140.58 | Å² | Ser | 78.16 | Å² |
| Glu | 134.61 | Å² | Thr | 101.67 | Å² |
| Gly | 32.28 | Å² | Trp | 210.89 | Å²20 |
| His | 147.00 | Å² | Tyr | 176.61 | Å² |
| Ile | 137.91 | Å² | Val | 114.14 | Å² |

Determination of surface exposed residues from structural models:

Surface accessibility and fractional ASA of side chains were calculated for each of the 50 model structures. The average value over the structural ensemble was used in the following. The N- and C-terminal residues of the FSH-α chain not included in the model are defined as having 100% side chain accessibility.

The following amino acid residues in hFSH-60 and hFSH-β, respectively, have more than 25% of their side chain exposed to the surface:

A1(a), P2(a), D3(a), V4(a), Q5(a), D6(a), P8(a), E9(a), T11(a), L12(a), Q13(a), E14(a), P16(a), F17(a), Q20(a), P21(a), G22(a), A23(a), P24(a), L26(a), M29(a), F33(a), R42(a), S43(a), K44(a), K45(a), T46(a), L48(a), V49(a), Q50(a), N52(a), V61(a), K63(a), S64(a), Y65(a), N66(a), R67(a), V68(a), T69(a), M71(a), G72(a), G73(a), F74(a), K75(a), N78(a), T80(a), A81(a), H83(a), C84(a), S85(a), T86(a), Y88(a), Y89(a), H90(a), K91(a), S92(a), N1(b), S2(b), E4(b), L5(b), T6(b), N7(b), I8(b), T9(b), K14(b), E15(b), E16(b), R18(b), F19(b), I21(b), S22(b), N24(b), Y31(b), Y33(b), R35(b), D36(b), L37(b), Y39(b), K40(b), D41(b), P42(b), A43(b), R44(b), P45(b), K46(b), I47(b), K49(b), K54(b), E55(b), L56(b), V57(b), Y58(b), E59(b), T60(b), V61(b), R62(b), P64(b), G65(b), A67(b), H68(b), H69(b), D71(b), L73(b), Y74(b), T75(b), T80(b), Q81(b), H83(b), G85(b), K86(b), D88(b), S89(b), D90(b), S91(b), D93(b), T95(b), V96(b), R97(b), G98(b), L99(b), G100(b), Y103(b), S105(b), F106(b), G107(b), E108(b), M109(b), K110(b), and E111(b).

The following amino acid residues have more than 50% of their side chain exposed to the surface:

A1(a), P2(a), D3(a), V4(a), Q5(a), D6(a), P8(a), E9(a), T11(a), Q13(a), E14(a), P16(a), F17(a), Q20(a), P21(a), G22(a), A23(a), K45(a), T46(a), L48(a), V49(a), Q50(a), N52(a), K63(a), S64(a), N66(a), R67(a), T69(a), G72(a), G73(a), K75(a), T86(a), Y89(a), H90(a), K91(a), S92(a), N1(b), N7(b), T9(b), E15(b), E16(b), R18(b), F19(b), N24(b), Y33(b), D41(b), P42(b), A43(b), R44(b), P45(b), K46(b), I47(b), K54(b), E55(b), V57(b), Y58(b), E59(b), R62(b), P64(b), G65(b), A67(b), H68(b), H69(b), D71(b), L73(b), T75(b), Q81(b), H83(b), K86(b), D88(b), S89(b), D90(b), S91(b), T95(b), R97(b), G98(b), L99(b), G100(b), Y103(b), S105(b), F106(b), G107(b), E108(b), M109(b), K110(b), and E111(b).

Determining distances between atoms

The distance between atoms is most easily determined using molecular graphics software, e.g., InsightII v. 98.0, MSI Inc.

### EXAMPLES

### Example 1

Construction of Plasmids for Expression of FSH

A gene encoding the human FSH-α subunit was constructed by assembly of synthetic oligonucleotides by PCR using methods similar to the ones described in Stemmer et al. (1995) Gene 164, pp. 49-53. The native FSH-α signal sequence was maintained in order to allow secretion of the gene product. The codon usage of the gene was optimised for high expression in mammalian cells. Furthermore, in order to achieve high gene expression, an intron (from pCI-Neo (Promega)) was included in the 5′ untranslated region of the gene. The synthetic gene was subcloned behind the CMV promoter in pcDNA3.1/Hygro (Invitrogen). The sequence of the resulting plasmid, termed pBvdH977, is given in SEQ ID NO:5 (FSH-α-coding sequence at position 1225 to 1572). Similarly, a synthetic gene encoding the wildtype human FSH-β subunit was constructed. Also in this construct, the native signal sequence was maintained in order to allow secretion, and the codon usage was optimised for high expression and an intron was included in the recipient vector (pcDNA3.1/Zeo (Invitrogen)). The sequence of the resulting FSH-β-containing plasmid, termed pBvdH1022, is given in SEQ ID NO:6 (FSH-β-coding sequence at position 1231 to 1617). A plasmid containing both the FSH-α and the FSH-β encoding synthetic genes was generated by subcloning the FSH-α containing NruI-PvuII fragment from pBvdH977 into pBvdH1022 linearized with NruI. The resulting plasmid, in which the FSH-α and FSH-β-expression cassettes are in direct orientation, was termed pBvdHII1100.

### Example 2

Expression of FSH in CHO Cells

FSH was expressed in Chinese Hamster Ovary (CHO) K1 cells, obtained from the American Type Culture Collection (ATCC, CCL 61).

For transient expression of FSH, cells were grown to 95% confluency in serum-containing media (MEMα with ribonucleotides and deoxyribonucleotides (Gibco/BRL Cat # 32571-028) containing 1:10 FBS (BioWhittaker Cat # 02-701F) and 1:100 penicillin and streptomycin (BioWhittaker Cat # BE17-602E), or Dulbecco's MEM/Nut.-mix F-12 (Ham) L-glutamine, 15 mM Hepes, pyridoxine-HCl (Life Technologies Cat # 11039-021) with the same additives. FSH-encoding plasmids were transfected into the cells using Lipofectamine 2000 (Life Technologies) according to the manufacturer's specifications. 24-48 hrs after transfection, culture media were collected, centrifuged and filtered through 0.22 μm filters to remove cells.

Stable clones expressing FSH were generated by transfection of CHO K1 cells with FSH-encoding plasmids followed by incubation of the cells in selective media (for instance one of the above media containing 0.5 mg/ml zeocin for cells transfected with plasmid pBvdH1100). Stably transfected cells were isolated and sub-cloned by limited dilution. Clones producing high levels of FSH were identified by ELISA (see below).

### Example 3

Large-scale Production of FSH in CHO Cells

The cell line CHO K1 1100-5, stably expressing human FSH, was passed 1:10 from a confluent culture and propagated as adherent cells in serum-containing medium Dulbecco's MEM/Nut.-mix F-12 (Ham) L-glutamine, 15 mM Hepes, pyridoxine-HCl (Life Technologies Cat # 11039-021), 1:10 FBS (BioWhittaker Cat # 02-701F), 1:100 penicillin and streptomycin (BioWhittaker Cat # BE17-602E) until confluence in a 10 layer cell factory (NUNC #165250). The media was then changed to serum-free media: Dulbecco's MEM/Nut.-mix F-12 (Ham) L-glutamine, 15 mM Hepes, pyridoxine-HCl (Life Technologies Cat # 11039-021) with the addition of 1:500 ITS-A (Gibco/BRL # 51300-044), 1:500 EX-CYTE VLE (Serological Proteins Inc. # 81-129-1) and 1:100 penicillin and streptomycin (BioWhittaker Cat # BE17-602E). Subsequently, every 24 h, culture media were collected and replaced with 1 fresh liter of the same serum-free media. The collected media was filtered through 0.22 μm filters to remove cells. Growth in cell factories was continued with daily harvests and replacements of the culture media until FSH yields dropped below 25% of the initial expression level (typically after 10-15 days).

### Example 4

Analysis of FSH Forms by Western Blotting and Isoelectric Focusing

The FSH content of samples was analysed by Western blotting: Proteins were separated by SDS-PAGE, and a standard Western blot was performed using rabbit anti human FSH (AHP519, Serotec) or mouse anti human FSH-β (MCA338, Serotec) as primary antibody, and an ImmunoPure Ultra Sensitive ABC Peroxidase Staining Kit (Pierce) for detection. Wild-type FSH produced as described above in Examples 1-3 was found to have the same mobility as FSH from references such as Puregon® (Organon) or Gonal-® (Serono).

For analysis of pI, samples were separated by on pH 3-7 IEF gels (NOVEX). After electrophoresis, proteins were blotted onto Immobilon-P (Millipore) membranes and a Western blot was performed as described above, using the same antibodies and detection kit. In accordance with published observations (see, for instance, Loumaye et al. (1998) Human Reprod. Update 4, 862-881), various FSH isoforms, mostly in the pH 4-5.2 range for wildtype FSH, were detected. This is due to heterogeneity in carbohydrate content, most importantly sialic acid.

### Example 5

Purification of FSH Wildtype and Variants

Three chromatographic steps have been employed to obtain highly purified FSH. First an anion exchanger step, then hydrophobic interaction chromatography (HIC) and finally an immunoaffinity step using an FSH-β specific monoclonal antibody.

Culture supernatants were prepared as described in Example 3. Filtered culture supernatants were concentrated 10 to 20 times by ultrafiltration (10 kD cut-off membrane), pH was adjusted to 8.0 and conductivity to 10-15 mS/cm, before application on a DEAE Sepharose (Pharmacia) anion exchanger column, previously equilibrated in ammonium acetate buffer (0.16 M, pH 8.0). The binding capacity for a 25 ml (2.6×4.7 cm) column was sufficient to bind at least 0.5 mg FSH. Semipurified FSH was recovered both in the unbound flow-through fraction as well as in the wash fraction using 0.16 M ammonium acetate, pH 8.0. The flow through and wash fractions were pooled and ammonium sulfate was added from a stock solution (4.5 M) to obtain a final concentration of 1.5 M (NH₄)₂SO₄. The pH was adjusted to 7.0.

The partially purified FSH was subsequently applied on a 25 ml butyl Sepharose (Pharmacia) HIC column. After application, the column was washed with at least 3 column volumes of 1.5 M (NH₄)₂SO₄, 20 mM ammonium acetate, pH 7 (until the absorbance at 280 nm reached baseline level) and FSH was eluted with 4 column volumes of buffer B (20 mM ammonium acetate, pH 7). FSH enriched fractions from the HIC step were pooled, concentrated and diafiltrated using Vivaspin 20 modules, 10 kD cut-off membrane (Vivascience), to a 50 mM sodium phosphate, 150 mM NaCl, pH 7.2.

For the third chromatographic step, an anti-FSH-β monoclonal antibody (RDI-FSH909, Research Diagnostics) was immobilized to CNBr-activated Sepharose (Pharmacia) using a standard procedure from the supplier. Approximately 1 mg antibody was coupled per ml resin. The immunoaffinity resin was packed in plastic columns and equilibrated with 50 mM sodium phosphate, 150 mM NaCl, pH 7.2 before application.

The buffer exchanged eluate from the butyl HIC step was applied on the antibody column by use of gravity flow. This was followed by several washing steps in 50 mM sodium phosphate solutions (0.5 M NaCl and 1 M NaCl, both pH 7.2). Elution was performed using either 1 M NH₃ or 0.6 M NH₃, 40% (v/v) isopropanol and the eluate was immediately neutralized with 1 M acetic acid to pH 6-8.

The purified FSH bulk product was concentrated and diafiltrated using Vivaspin 20 modules, 10 kD cut-off membrane (Vivascience), to a 50 mM sodium phosphate, 150 mM NaCl, pH 7.2. For subsequent storage, BSA was added to 0.1% (w/v) and the purified FSH was microfiltrated using a 0.22 μm filter prior to storage at −80° C.

SDS-PAGE, run under non-dissociating conditions (without boiling), showed wildtype FSH migrating as an apparant 42±3 kDa band, slightly diffuse due to heterogeneity in the attached carbohydrates. The purity was about 80-90%. N-terminal sequencing showed that the α-chain had the expected N-terminal sequence starting with residue 1 (SEQ ID NO:2) and the β-chain starting with residue 3 (SEQ ID NO:4). These N-terminal sequences have been found previously for recombinant FSH produced in CHO cells (Olijve, W. et al. (1996) Mol. Hum. Reprod. 2, 371-382).

### Example 6

FSH in Vitro Activity Assay

6.1 FSH assay Outline

It has previously been published that activation of the FSH receptor by FSH leads to an increase in the intracellular concentration of cAMP. Consequently, transcription is activated at promoters containing multiple copies of the cAMP response element (CRE). It is thus possible to measure FSH activity by use of a CRE luciferase reporter gene introduced into CHO cells expressing the FSH receptor. 6.2 Construction of a CHO FSH-R/CRE-luc cell line

Stable clones expressing the human FSH receptor were produced by transfection of CHO K1 cells with a plasmid containing the receptor cDNA inserted into pcDNA3 (Invitrogen) followed by selection in media containing 600 μg/ml G418. Using a commercial cAMP-SPA RIA (Amersham), clones were screened for the ability to respond to FSH stimulation. On the basis of these results, an FSH receptor-expressing CHO clone was selected for further transfection with a CRE-luc reporter gene. A plasmid containing the reporter gene with 6 CRE elements in front of the Firefly luciferase gene was co-transfected with a plasmid conferring Hygromycin B resistance. Stable clones were selected in the presence of 600 μ/ml G418 and 400 μg/ml Hygromycin B. A clone yielding a robust luciferase signal upon stimulation with FSH (EC₅₀-0.01 IU/ml) was obtained. This CHO FSH-R/CRE-luc cell line was used to measure the activity of samples containing FSH. 6.3 FSH luciferase assay

To perform activity assays, CHO FSH-R/CRE-luc cells were seeded in white 96 well culture plates at a density of about 15,000 cells/well. The cells were in 100 μl DMEM/F-12 (without phenol red) with 1.25% FBS. After incubation overnight (at 37° C., 5% CO₂), 25 μl of sample or standard diluted in DMEM/F-12 (without phenol red) with 10% FBS was added to each well. The plates were further incubated for 3 hrs followed by addition of 125 μl LucLite substrate (Packard Bioscience). Subsequently, plates were sealed and luminescence was measured on a TopCount luminometer (Packard) in SPC (single photon counting) mode.

### Example 7

FSH Elisa

The concentration of FSH in samples was quantified by use of a commercial immunoassay (DRG Instruments GmbH, Marburg, Germany). DRG FSH EIA is a solid phase immunosorbent assay (ELISA) based on the sandwich principle. The microtiter wells are coated with a monoclonal antibody directed towards a unique antigenic site on the FSH-β subunit. An aliquot of FSH-containing sample (diluted in H₂O with 0.1% BSA) and an anti-FSH antiserum conjugated with horseradish peroxidase are added to the coated wells. After incubation, unbound conjugate is washed off with water. The amount of bound peroxidase is proportional to the concentration of FSH in the sample. The intensity of colour developed upon addition of substrate solution is proportional to the concentration of FSH in the sample.

### Example 8

Animal Studies

The pharmakinetic profile of FSH and variant forms was determined as follows: Immature 26-27 days old female Sprague-Dawley rats were injected i.v. with 3-4 μg FSH, and blood samples were taken at various time-points after injection. FSH concentrations in serum samples were determined by ELISA, as described in Example 7. In vivo bioactivity of wildtype recombinant FSH and variant forms can be evaluated by the ovarian weight augmentation assay (Steelman and Pohley (1953) Endocrinology 53, 604-616). Furthermore, the ability of FSH and variant forms to stimulate maturation of follicles in laboratory animals can be detected with e.g., ultrasound equipment.

### Example 9

Construction and Abalysis of a Variant Form of FSH Containing Two N-linked Glycosylations at the N-terminus of the α Subunit

A construct encoding a modified form of FSH-α, having two additional sites for N-linked glycosylation at its N-terminus was generated by site-directed mutagenesis using standard DNA techniques known in the art. A DNA fragment encoding the sequence Ala-Asn-Ile-Thr-Val-Asn-He-Thr-Val was inserted immediately upstream of the mature FSH-α sequence in pBvdH977. The sequence of the resulting plasmid, termed pBvdH1163, is given in SEQ ID NO:7 (modified FSH-α-encoding sequence at position 1225 to 1599). A plasmid encoding both subunits was constructed by subcloning the FSH-containing NruI-PvuII fragment from pBvdH1163 into pBvdH1022 (Example 1), which had been linearized with PvuII. The resulting plasmid was termed pBvdH1208.

For expression of the variant form of FSH containing two N-linked glycosylations at the N-terminus of the α subunit (termed FSH1208), CHO K1 cells were transfected with pBvdH1208 or co-transfected with a combination of pBvdH1163, encoding the modified α subunit and pBvdH1022, encoding the wildtype β subunit. Transient expressions, isolation of stable expression clones, and large-scale production of FSH1208 were performed as described for wildtype FSH in Examples 2 and 3.

Western blotting showed that FSH1208 has a larger molecular mass than wildtype FSH, indicating that the introduction of acceptor sites for N-linked glycosylation at the N-terminus of the α subunit indeed leads to hyperglycosylation of FSH. Isoelectric focusing demonstrated that the FSH forms in the FSH1208 samples were found in a lower pI range than wildtype FSH produced as described in Examples 1-4. Thus, the pH interval for FSH1208 isoforms was about 3.0-4.5 versus about 4.0-5.2 for wildtype FSH. This indicated that FSH1208 molecules are on average more negatively charged than the wild type, which is attributed to the presence of additional sialic acid residues.

FSH1208 was purified and characterized as described in Examples 4 and 5. SDS-PAGE, run under non-dissociating conditions (without boiling), showed FSH1208 migrating as an apparent 55±5 kDa band, slightly diffuse due to heterogeneity in the attached carbohydrates. The purity was about 80-90%. N-terminal sequencing showed that while the β-chain had the same N-terminal sequence as wildtype FSH, the sequence of α-chain was in agreement with this subunit carrying the expected N-terminal extension ANITVNITV, in which both asparagines residues are glycosylated.

The specific activity of FSH1208 was determined by measurement of the in vitro bioactivity (FSH luciferase assay, Example 6.3) and the FSH content of the samples (FSH ELISA, Examples). The specific activity of FSH1208 was found to be about one-third of that of the wildtype reference.

A pharmacokinetic study performed as described in Example 8 showed that 24 hours after injection of equal amounts of wildtype FSH and FSH1208, the sera of FSH1208-treated animals contained more than 10 fold more remaining immunoreactive material than the sera from animals treated with wildtype FSH.

### Example 10

Construction and Analysis of other FSH Variants Containing Additional Glycosylation Sites

Plasmids encoding variant forms of FSH-α and FSH-β containing additional sites for N-linked glycosylation were generated by site-directed mutagenesis using standard DNA techniques known in the art. The following amino acid substitutions and/or insertions were generated:

FSH1147: Amino acid Tyr58 of mature FSH-β altered to Asn

FSH1349: N-terminus of mature FSH-α altered from APD QDC . . . to: APNDTVNFT QDC . . .

FSH1354: N-terminus of mature FSH-β altered from NS CEL . . . to: NSNITVNITV CEL . . .

Plasmids encoding the variant forms were transiently expressed in CHO K1 cells as described in Example 2. Plasmids encoding FSH-α variants were co-transfected with a plasmid encoding wild-type FSH-β and vice versa.

Western and isoelectric focusing were performed on culture media samples as described in Example 4. The variant forms had higher molecular weights than the wild-type, indicating that the additional acceptor sites for N-linked glycosylation had indeed been glycosylated. Furthermore, isoelectric focusing showed that the different isoforms of the three FSH variants were spread over a lower pI range than the wildtype. This strongly suggests that the variant forms had a higher sialic acid content than the wildtype.

In vitro FSH activities of the resulting media samples were analysed as described in Example 6.3. All three variant forms were able to stimulate the CHO FSH-R/CRE-luc cells, indicating that these variant FSH forms have retained significant FSH activity.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above can be used in various combinations. All publications, patents, patent applications, or other documents cited in this application are incorporated by reference in their entirety for all purposes to the same extent as if each individual publication, patent, patent

What is claimed is:

1. A heterodimeric polypeptide conjugate exhibiting FSH activity, comprising
i) a dimeric polypeptide comprising an FSH-α subunit and an FSH-β subunit, wherein at least one of said FSH-α and FSH-β subunits differs from the corresponding wildtype subunit in that at least one amino acid residue acid residue comprising an attachment group for a non-polypeptide moiety has been introduced or removed, and
ii) at least one non-polypeptide moiety bound to an attachment group of at least one of said subunits.

2. The conjugate of claim 1, wherein the amino acid sequence of at least one of said FSH-α and FSH-β subunits differs from that of the corresponding wildtype subunit in that an amino acid residue comprising an attachment group for the non-polypeptide moiety has been introduced.

3. The conjugate of claim 2, wherein the introduced attachment group is selected from the group consisting of an N-glycosylation site, an O-glycosylation site, and an attachment group for a polymer molecule, a lipophilic compound, a carbohydrate moiety or an organic derivatizing agent.

4. The conjugate of claim 1, comprising at least one PEG molecule attached to an attachment group of at least one of the subunits.

5. The conjugate of claim 1, comprising at least one introduced N-glycosylation site, and further comprising at least one PEG molecule attached to an attachment group of at least one of the subunits.

6. The conjugate of claim 5, wherein said at least one PEG molecule is bound to the N-terminal of at least one of the subunits.

7. The conjugate of claim 1, wherein the amino acid sequence of the FSH-α subunit differs from that of wildtype human FSH-α.

8. The conjugate of claim 1, wherein the amino acid sequence of the FSH-β subunit differs from that of wildtype human FSH-β.

9. A heterodimeric polypeptide conjugate exhibiting FSH activity, comprising
i) a dimeric polypeptide comprising an FSH-α subunit and an FSH-β subunit, wherein the amino acid sequence of at least one of said FSH-α and FSH-β subunits differs from that of the corresponding wildtype subunit in that at least one N-glycosylation site has been introduced, and
ii) at least one oligosaccharide moiety bound to an N-glycosylation site of at least one of said subunits.

10. The conjugate of claim 9, wherein at least one N-glycosylation site has been introduced into the FSH-α subunit by a mutation selected from the group consisting of P2(a)N+V4(a)S, P2(a)N+V4(a)T, D3(a)N+Q5(a)S, D3(a)N+Q5(a)T, V4(a)N+D6(a)S, V4(a)N+D6(a)S, D6(a)N+P8(a)S, D6(a)N+P8(a)T, E9(a)N+T11(a)S, E9(a)N, T11(a)N+Q13(a)S, T11(a)N+Q13(a)T, L12(a)N+E14(a)S, L12(a)N+E14(a)T, E14(a)N+P16(a)S, E14(a)N+P16(a)T, P16(a)N+F18(a)S, P16(a)N+F18(a)T, F17(a)N, F17(a)N+S19(a)T, G22(a)N+P24(a)S, G22(a)N+P24(a)T, P24(a)N+L26(a)S, P24(a)N+L26(a)T, F33(a)N+R35(a)S, F33(a)N+R35(a)T, R42(a)N+K44(a)S, R42(a)N+K44(a)T, S43(a)N+K45(a)S, S43(a)N+K45(a)T, K44(a)N+T46(a)S, K44(a)N, K45(a)N+M47(a)S, K45(a)N+M47(a)T, T46(a)N+L48(a)S, T46(a)N+L48(a)T, L48(a)N+Q50(a)S, 148(a)N+Q50(a)T, V49(a)N+H51(a)S, V49(a)N+K51(a)T, Q50(a)N+N52(a)S, Q50(a)N+N52(a)T, V61(a)N+K63(a)S, V61(a)N+K63(a)T, K63(a)N+Y65(a)S, K63(a)N+Y65(a)T, S64(a)N+N66(a)S, S64(a)N+N66(a)T, Y65(a)N+R67(a)S, Y65(a)N+R67(a)T, V68(a)S, V68(a)T, R67(a)N+T69(a)S, R67(a)N, T69(a)N+M71(a)S, T69(a)N+M71(a)T, M71(a)N+G73(a)S, M71(a)N+G73(a)T, G72(a)N+F74(a)S, G72(a)N+F74(a)T, G73(a)N+K75(a)S, G73(a)N+K75(a)T, F74(a)N+V76(a)S, F74(a)N+V76(a)T, K75(a)N+E77(a)S, K75(a)N+E77(a)T, A81(a)N+II83(a)S, A81(a)N+H83(a)T, H83(a)N, T86(a)N+Y88(a)S, T86(a)N+Y88(a)T, Y88(a)N+II90(a)S, Y88(a)N+H90(a)T, Y89(a)N+K91(a)S, Y89(a)N+K91(a)T, H90(a)N and H90(a)N+S92(a)T.

11. The conjugate of claim 9, wherein at least one N-glycosylation site has been introduced into the FSH-β subunit by a mutation selected from the group consisting of S2(b)N+E4(b)S, S2(b)N+E4(b)T, E4(b)N+T6(b)S, E4(b)N, L5(b)N+N7(b)S, L5(b)N+L7(b)T, T6(b)N+I8(b)S, T6(b)N+I8(b)T, I8(b)N+I10(b)S, I8(b)N+I10(b)T, T9(b)N+A11(b)S, T9(b)N+A11(b)T, K14(b)N+E16(b)S, K14(b)N+E16(b)T, F19(b)N+I21(b)S, F19(b)N+I21(b)T, I21(b)N+I23(b)S, I21(b)N+I23(b)T, S22(b)N+N24(b)S, S22(b)N+N24(b)T, Y31(b)N+Y33(b)S, Y31(b)N+Y33(b)T, Y33(b)N+R35(b)S, Y33(b)N+R35(b)T, R35(b)N+L37(b)S, R35(b)N+L37(b)T, D36(b)N+V38(b)S, D36(b)N+V38(b)T, L37(b)N+Y39(b)S, L37(b)N+Y39(b)T, K40(b)N+P42(b)S, K40(b)N+P42(b)T, A43(b)N+P45(b)S, A43(b)N+P45(b)T, P45(b)N+I47(b)S, P45(b)N+I47(b)T, K46(b)N+Q48(b)S, K46(b)N+Q48(b)T, I47(b)N+K49(b)S, I47(b)N+K49(b)T, K54(b)N+L56(b)S, K54(b)N+L56(b)T, E55(b)N+V57(b)S, E55(b)N+V57(b)T, L56(b)N+Y58(b)S, L56(b)N+Y58(b)T, V57(b)N+E59(b)S, V57(b)N+E59(b)T, Y58(b)N+T60(b)S, Y58(b)N, E59(b)N+V61(b)S, E59(b)N+V61(b)T, T60(b)N+R62(b)S, T60(b)N+R62(b)T, R62(b)N+P64(b)S, R62(b)N+P64(b)T, G65(b)N+A67(b)S, G65(b)N+A67(b)T, A67(b)N+H69(b)S, A67(b)N+H69(b)T, H68(b)N+A70(b)S, H68(b)N+A70(b)T, H69(b)N+D71(b)S, H69(b)N+D71(b)T, D71(b)N+L73(b)S, D71(b)N+L73(b)T, L73(b)N+T75(b)S, L73(b)N, T75(b)N+P77(b)S, T75(b)N+P77(b)T, H83(b)N+G85(b)S, H83(b)N+G85(b)T, K86(b)N+D88(b)S, K86(b)N+D88(b)T, D88(b)N+D90(b)S, D88(b)N+D90(b)T, S89(b)N, S89(b)N+S91(b)T, D90(b)N+T92(b)S, D90(b)N, S91(b)N+D93(b)S, S91(b)N+D93(b)T, D93(b)N+T96(b)S, D93(b)N, T95(b)N+R97(b)S, T95(b)N+R97(b)T, V96(b)N+G98(b)S, V96(b)N+G98(b)T, R97(b)N+L99(b)S, R97(b)N+L99(b)T, L99(b)N+P101(b)S, L99(b)N+P101(b)T, Y103(b)N, Y103(b)N+S105(b)T, S105(b)N+G107(b)S, S105(b)N+G107(b)T, F106(b)N+E108(b)S, F106(b)N+E108(b)T, G107(b)N+M109(b)S, G107(b)N+M109(b)T, E108(b)N+K110(b)S, E108(b)N+K110(b)T, M109(b)N+E111(b)S, and M109(b)N+E111(b)T.

12. The conjugate of claim 9, wherein at least one of the FSH-α and FSH-β subunits comprises at least one N- or C-terminal peptide addition comprising at least one N-glycosylation site.

13. The conjugate of claim 9, which further comprises at least one non-polypeptide moiety different from an N- or O-linked oligosaccharide moiety bound to an attachment group of the polypeptide.

14. The conjugate of claim 9, wherein the amino acid sequence of at least one of said FSH-α and FSH-β subunits further differs from that of the corresponding wildtype subunit in that at least one naturally occurring N-glycosylation site has been removed.

15. A heterodimeric polypeptide conjugate exhibiting FSH activity, comprising a dimeric polypeptide comprising an FSH-α subunit and an FSH-β subunit, wherein at least one of said FSH-α and FSH-β subunits comprises a polymer molecule bound to the N-terminal thereof.

16. The conjugate of claim 15, wherein the polymer molecule is polyethylene glycol.

17. The conjugate of claim 15, wherein at least one of said FSH-α and FSH-β subunit comprises, relative to the corresponding wildtype human subunit, at least one introduced amino acid residue comprising an attachment group for the polymer molecule, and/or wherein at least one amino acid residue comprising an attachment group for a polymer molecule has been removed.

18. A heterodimeric polypeptide conjugate exhibiting FSH activity, comprising a dimeric polypeptide comprising FSH-α and FSH-β subunits, wherein at least one of said FSH-α and FSH-β subunits comprises, relative to the corresponding wildtype subunit, at least one introduced N- or O-glycosylation site at the N-terminal thereof, said at least one introduced glycosylation site being glycosylated.

19. The conjugate of claim 18, wherein said at least one introduced N- or O-glycosylation site is part of an N-terminal peptide addition.

20. The conjugate of claim 1, wherein the FSH-α subunit comprises hFSH-α having the sequence shown in SEQ ID NO:2, or the FSH-β subunit comprises hFSH-β having the sequence shown in SEQ ID NO:4.

21. The conjugate of claim 1, wherein the amino acid sequence of the FSH-α and/or FSH-β subunit differs in 1-20 amino acid residues from that of the corresponding wildtype sequence.

22. The conjugate of claim 1, which has an increased functional in vivo half-life and/or serum half-life as compared to hFSH.

23. The conjugate of claim 1, wherein the FSH-α subunit and the FSH-β subunit are linked by a peptide bond or a peptide linker to form a single-chain polypeptide.

24. A composition comprising a conjugate according to claim 1 and at least one pharmaceutically acceptable carrier or excipient.

25. A composition comprising a conjugate according to claim 9 and at least one pharmaceutically acceptable carrier or excipient.

26. A composition comprising a conjugate according to claim 15 and at least one pharmaceutically acceptable carrier or excipient.

27. A composition comprising a conjugate according to claim 18 and at least one pharmaceutically acceptable carrier or excipient.

28. A method of treating an infertile mammal, comprising administering to a mammal in need thereof an effective amount of a conjugate according to claim 1.

29. A method of treating an infertile mammal, comprising administering to a mammal in need thereof an effective amount of a conjugate according to claim 9.

30. A method of treating an infertile mammal, comprising administering to a mammal in need thereof an effective amount of a conjugate according to claim 15.

31. A method of treating an infertile mammal, comprising administering to a mammal in need thereof an effective amount of a conjugate according to claim 18.

32. A modified FSH-α polypeptide subunit having an amino acid sequence that differs from that of the wildtype hFSH-α subunit in that at least one amino acid residue comprising an attachment group for a non-polypeptide moiety has been introduced.

33. A modified FSH-β polypeptide subunit having has an amino acid sequence that differs from that of the wildtype hFSH-β subunit in that at least one amino acid residue comprising an attachment group for a non-polypeptide moiety has been introduced.

34. A nucleotide sequence encoding a modified FSH-α polypeptide subunit having an amino acid sequence that differs from that of the wildtype hFSH-α subunit in that at least one amino acid residue comprising an attachment group for a non-polypeptide moiety has been introduced; and/or encoding a modified FSH-β polypeptide subunit having has an amino acid sequence that differs from that of the wildtype hFSH-β subunit in that at least one amino acid residue comprising an attachment group for a non-polypeptide moiety has been introduced.

35. An expression vector comprising a nucleotide sequence according to claim 34.

36. A host cell comprising a nucleotide sequence according to claim 34.

37. A method for producing a recombinant heterodimeric FSH protein, comprising subjecting a host cell according to claim 34 comprising a nucleotide sequence encoding an FSH-α subunit and an FSH-β subunit to cultivation under conditions conducive for expression of said subunits.

38. The method of claim 37, wherein the host cell is a eukaryotic cell capable of in vivo glycosylation, and the amino acid sequence of at least one of said FSH-α and FSH-β subunits differs from the sequence of the corresponding wildtype subunit in that at least one N-glycosylation site has been introduced.

39. The method of claim 38, further comprising subjecting the heterodimeric protein to in vitro conjugation to a non-polypeptide moiety.


SEQUENCE LISTING (30 sequences)

SEQ ID NO: 1 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 3 - protein, 129 aa, Homo sapiens
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 4 - protein, 111 aa, Homo sapiens
NSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 5 - DNA, 6186 bp, Homo sapiens
  CDS (1225)..(1572)
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaagcttggtaccgagctcggatccgccaccatggactactaccgcaagtacgccgccatcttcctggtgaccctgagcgtgttcctgcacgtgctgcacagcgcccccgacgtgcaggactgccccgagtgcaccctgcaggagaaccccttcttcagccagcccggcgcccccatcctgcagtgcatgggctgctgcttcagccgcgcctaccccacccccctgcgcagcaagaagaccatgctggtgcagaagaacgtgaccagcgagagcacctgctgcgtggccaagagctacaaccgcgtgaccgtgatgggcggcttcaaggtggagaaccacaccgcctgccactgcagcacctgctactaccacaagagctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 6 - DNA, 5651 bp, Homo sapiens
  CDS (1231)..(1617)
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaagcttggtaccgagctcggatctatcgatgccaccatggagaccctgcagttcttcttcctgttctgctgctggaaggccatctgctgcaacagctgcgagctgaccaacatcaccatcgccatcgagaaggaggagtgccgcttctgcatcagcatcaacaccacctggtgcgccggctactgctacacccgcgacctggtgtacaaggaccccgcccgccccaagatccagaagacctgcaccttcaaggagctggtgtacgagacggtccgggtgcccggctgcgcccaccacgccgacagcctgtacacctaccccgtggccacccagtgccactgcggcaagtgcgacagcgacagcaccgactgcaccgtgcgcggcctgggccccagctactgcagcttcggcgagatgaaggagtaactcgagactagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 6213 bp, Homo sapiens
  CDS (1225)..(1599)
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaagcttggtaccgagctcggatccgccaccatggactactaccgcaagtacgccgccatcttcctggtgaccctgagcgtgttcctgcacgtgctgcacagcgccaacatcaccgttaacatcaccgtggcccccgacgtgcaggactgccccgagtgcaccctgcaggagaaccccttcttcagccagcccggcgcccccatcctgcagtgcatgggctgctgcttcagccgcgcctaccccacccccctgcgcagcaagaagaccatgctggtgcagaagaacgtgaccagcgagagcacctgctgcgtggccaagagctacaaccgcgtgaccgtgatgggcggcttcaaggtggagaaccacaccgcctgccactgcagcacctgctactaccacaagagctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - protein, 8 aa, Artificial Sequence
  Description of Artificial Sequence Synthetic peptide
NSTQNATA

SEQ ID NO: 9 - protein, 14 aa, Artificial Sequence
  Description of Artificial Sequence Synthetic peptide
ANLTVRNLTRNVTV

SEQ ID NO: 10 - protein, 9 aa, Artificial Sequence
  Description of Artificial Sequence Synthetic peptide
ANITVNITV

SEQ ID NO: 11 - protein, 7 aa, Artificial Sequence
  Description of Artificial Sequence Synthetic peptide
NDTVNFT

SEQ ID NO: 12 - protein, 8 aa, Artificial Sequence
  Description of Artificial Sequence Synthetic peptide
NITVNITV

SEQ ID NO: 13 - protein, 6 aa, Artificial Sequence
  Description of Artificial Sequence Synthetic peptide
AATPAP

SEQ ID NO: 14 - protein, 6 aa, Artificial Sequence
  Description of Artificial Sequence Synthetic peptide
HHHHHH

SEQ ID NO: 15 - protein, 8 aa, Artificial Sequence
  Description of Artificial Sequence Synthetic peptide
MKHHHHHH

SEQ ID NO: 16 - protein, 10 aa, Artificial Sequence
  Description of Artificial Sequence Synthetic peptide
MKHHAHHQHH

SEQ ID NO: 17 - protein, 14 aa, Artificial Sequence
  Description of Artificial Sequence Synthetic peptide
MKHQHQHQHQHQHQ

SEQ ID NO: 18 - protein, 15 aa, Artificial Sequence
  Description of Artificial Sequence Synthetic peptide
MKHQHQHQHQHQHQQ

SEQ ID NO: 19 - protein, 10 aa, Artificial Sequence
  Description of Artificial Sequence Synthetic peptide
EQKLISEEDL

SEQ ID NO: 20 - protein, 8 aa, Artificial Sequence
  Description of Artificial Sequence Synthetic peptide
DYKDDDDK

SEQ ID NO: 21 - protein, 9 aa, Artificial Sequence
  Description of Artificial Sequence Synthetic peptide
YPYDVPDYA

SEQ ID NO: 22 - protein, 5 aa, Artificial Sequence
  Description of Artificial Sequence Synthetic peptide
GGGGS

SEQ ID NO: 23 - protein, 129 aa, Homo sapiens
METLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 24 - protein, 125 aa, Homo sapiens
MDYYRKYAAIFLVTLSVFLHVLHSANITVNITVAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 25 - protein, 6 aa, Homo sapiens
APDQDC

SEQ ID NO: 26 - protein, 12 aa, Artificial Sequence
  Description of Artificial Sequence Synthetic peptide
APNDTVNFTQDC

SEQ ID NO: 27 - protein, 13 aa, Artificial Sequence
  Description of Artificial Sequence Synthetic peptide
NSNITVNITVCEL

SEQ ID NO: 28 - protein, 196 aa, Homo sapiens
QDCPECTLQENPFFSQPGAPILQCMGCCFSRAYPTPLRSKKTMLVQKNVTSESTCCVAKSYNRVTVMGGFKVENHTACHCSTCYYNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 29 - protein, 196 aa, Homo sapiens
TQDCPECTLQENPFFSQPGAPILQCMGCCFSRAYPTPLRSKKTMLVQKNVTSESTCCVAKSYNRVTVMGGFKVENHTACHCSTCYYKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCD

SEQ ID NO: 30 - protein, 195 aa, Homo sapiens
QDCPECTLQENPFFSQPGAPILQCMGCCFSRAYPTPLRSKKTMLVQKNVTSESTCCVAKSYNRVTVMGGFKVENHTACHCSTCYYKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCD